(19) United States
(12) Patent Application Publication
(10) Pub. No.: US 2005/0008892 A1
(43) Pub. Date: Jan. 13, 2005

(54) Fluorescent compositions comprising diketopyrrolopyrroles

(76) Inventors: Hiroshi Yamamoto, Nishinomiya-shi (JP); Norihisa Dan, Yawata-shi (JP)

Correspondence Address: CIBA SPECIALTY CHEMICALS CORPORATION;PATENT DEPARTMENT, 540 WHITE PLAINS RD, P O BOX 2005, TARRYTOWN, NY, 10591-9005, US

(21) Appl. No.: 10/501,573

(22) PCT Filed: Jan. 23, 2003
(86) PCT No.: PCT/EP03/00650

(30) Foreign Application Priority Data
Feb. 1, 2002 (EP) 0245067.6
Sep. 12, 2002 (EP) 0245796.0

(51) Int. Cl. C09K 11/06; H05B 33/14; C07D 487/02

(52) U.S. Cl. 428/690; 428/917; 252/301.16; 313/504; 313/506; 548/454

(57) ABSTRACT

The present invention relates to compositions comprising a guest chromophore and a host chromophore, wherein the absorption spectrum of the guest chromophore overlaps with the fluorescence emission spectrum of the host chromophore, wherein the host chromophore is a diketopyrrolopyrrole having an absorption peak at 500 to 720 nm, preferably 500 to 600 nm, most preferred 520 to 580 nm and wherein the guest chromophore is a diketopyrrolopyrrole having an absorption peak at 500 to 720 nm, preferably 500 to 600 nm, most preferred 520 to 580 nm and their use for the preparation of inks, colorants, pigmented plastics for coatings, non-impact-printing material, color filters, cosmetics, polymeric ink particles, toners, dye lasers and electroluminescent devices. A luminescent device comprising a composition according to the present invention is high in the efficiency of electrical energy utilisation and high in luminance.

The present invention relates to fluorescent compositions comprising a guest chromophore and a host chromophore, wherein the absorption spectrum of the guest chromophore overlaps with the fluorescence emission spectrum of the host chromophore, wherein the host chromophore is a diketopyrrolopyrrole having a photoluminescence emission peak at 500 to 720 nm, preferably 500 to 600 nm, most preferred 520 to 580 nm and wherein the guest chromophore is a diketopyrrolopyrrole having an absorption peak at 500 to 720 nm, preferably 500 to 600 nm, most preferred 520 to 580 nm and their use for the preparation of inks, colorants, pigmented plastics for coatings, non-impact-printing material, color filters, cosmetics, polymeric ink particles, toners, dye lasers and electroluminescent devices. A luminescent device comprising a composition according to the present invention is high in the efficiency of electrical energy utilisation and high in luminance.

It is presently common to prepare organic electroluminescent (“EL”) devices which contain an organic fluorescent substance by a vacuum evaporation process, e.g. described in Appl. Phys. Lett., 51, 913 (1987). In general, two types of such vacuum evaporation processes are applied according to the constitution of light emitting material: a one-component type process and a two-component type (or “Host-Guest type” or “binary system”) process (e.g. described in J. Appl. Phys., 65, 3610 (1989)).

For emitting a light of red, green or blue color in a one-component system, the light emitting materials themselves have to emit an intense fluorescence of red, green or blue color. Further, a vacuum evaporation process has to give a deposited film of uniform quality, and the film thus formed has to be endowed with appropriate (“carrier”) mobility for positive holes and/or electrons i.e. properties of a semiconductor.

Numerous materials emitting light in the green- or blue-colored region are known.

JP-B2 2,749,407 (Pioneer Electron Corp. & Nippon Kayaku Co. Ltd.) describes as a light emitting material N,N′-bis(2,5-di-tert.-butylphenyl)-3,4,9,10-perylenedicarboximide. However, its luminance is as low as 27 cd/m², which is insufficient for commercial applications.

JP-A2 2,296,891 (Ricoh) claims an electroluminescent element comprising a positive electrode, a negative electrode and one organic compound layer or a plurality of organic compound layers held between the positive and negative electrodes, but no hole transporting substance. At least one layer of said organic compound layers is a layer containing a pyrrolopyrrole compound represented by the following formula II″ 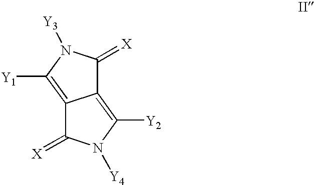
wherein Y₁ and Y₂ independently from each other represent a substituted or unsubstituted alkyl, cycloalkyl or aryl group, Y₃ and Y₄ independently represent a hydrogen atom or a substituted or unsubstituted alkyl or aryl group, and X represents an oxygen or a sulfur atom. Only four compounds are mentioned explicitly, namely wherein X stands for oxygen in all cases, and wherein (a) Y₃=Y₄=methyl and Y₁=Y₂=p-tolyl, (b) Y₃=Y₄=methyl and Y₁=Y₂=hydrogen, (c) Y₃=Y₄=hydrogen and Y₁=Y₂=p-tolyl, and (d) Y₃=Y₄=Y₁=hydrogen and Y₂=p-chlorophenyl. However, according to JP-A2 5,320,633 (see below), a follow-up study of the same inventors revealed that an emission of light is only observed, if the DPP-compounds II″ are used together with other compounds. This observation is supported by comparative example 2 of JP-A2 5,320,633, which shows that no emission is observed, if DPP II″ is used alone, i.e. without the addition of tris(8-hydroxyquinolinato)aluminium (“Alq₃”).

JP-A2 5,320,633 (Sumitomo) claims an organic EL device having a light emitting layer comprising a light emitting material in an amount of 0.005 to 15 parts by weight of a DPP compound between a pair of electrodes, wherein at least one electrode being transparent or semi-transparent. Although the main claim is silent about the use of Alq₃, it is clear from the specification and the examples, especially from comparative example 2, that Alq₃ is an essential feature in the claimed EL element or device.

JP-A2 9003448 (Toyo Ink) claims an organic EL element having between a pair of electrodes a luminous layer containing a DPP compound as electron-transporting material or an organic compound thin film layer including a luminous layer and an electron-injecting layer wherein the electron-injecting layer contains a DPP compound as the electron-transporting material. In addition, another EL element further comprising a hole-injecting layer is claimed. The disadvantage of the claimed EL devices is that according to the examples always Alq₃ and a phenanthrene diamine (as hole-injecting material) have to be used.

EP-A 499,011 describes electroluminescent devices comprising DPP-compounds. Particularly, in example 1 the DPP-derivative of formula III′ 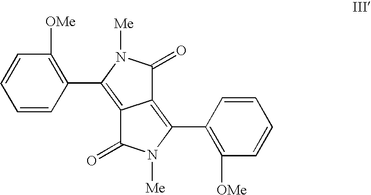
is disclosed.

WO 98/33862 describes the use of the DPP-compound of formula IV′ 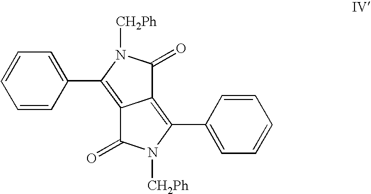
as a guest molecule in electroluminescent devices.

EP-A-1087005 relates to fluorescent diketbpyrrolopyrroles (“DPPs”) of the formula I′ 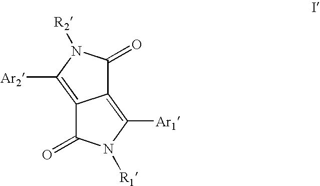
wherein R_{1′} and R_{2′}, independently from each other, stand for C₁-C₂₅-alkyl, allyl which can be substituted one to three times with C₁-C₃alkyl or Ar_{3′}, —CR_{3′}R_{4′}—(CH₂)ₘ—Ar_{3′}, wherein R_{3′} and R_{4′} independently from each other stand for hydrogen or C₁-C₄alkyl, or phenyl which can be substituted on to three times with C₁-C₃ alkyl, Ar_{3′} stands for phenyl or 1- or 2-naphthyl which can be substituted one to three times with C₁-C₈alkyl, C₁-C₈alkoxy, halogen or phenyl, which can be substituted with C₁-C₈alkyl or C₁-C₈alkoxy one to three times, and m′ stands for 0, 1, 2, 3 or 4, and wherein C₁-C₂₅-alkyl or —CR_{3′}R_{4′}—(CH₂)ₘ—Ar_{3′}, preferably C₁-C₂₅-alkyl, can be substituted with a functional group capable of increasing the solubility in water such as a tertiary amino group, —SO₃⁻, or PO₄²⁻, Ar₁ and Ar₂, independently from each other, stand for 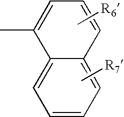
wherein R₆′ and R₇′, independently from each other, stand for hydrogen, C₁-C₆alkyl, —NR₈′R₉′, —OR₁₀′, —S(O)ₙR₈′, —Se(O)ₙR₈′, or phenyl, which can be substituted one to three times with C₁-C₈alkyl or C₁-C₈alkoxy, but do not stand simultaneously for hydrogen, wherein R_{8′} and R_{9′}, independently from each other, stand for hydrogen, C₁-C₂₅-alkyl, C₅-C₁₂-cycloalkyl, —CR_{3′}R_{4′}—(CH₂)_{m′}—Ph, R_{10′}, wherein R₁₀′ stands for C₆-C₂₄-aryl, or a saturated or unsaturated heterocyclic radical comprising five to seven ring atoms, wherein the ring consists of carbon atoms and one to three hetero atoms selected from the group consisting of nitrogen, oxygen and sulfur, wherein Ph, the aryl and heterocyclic radical can be substituted one to three times with C₁-C₈alkyl, C₁-C₈alkoxy, or halogen, or R_{8′} and R_{9′} stand for —C(∩)R_{10′}, wherein R_{11′} can be C₁-C₂₅-alkyl, C₅-C₁₂-cycloalkyl, R_{10′}, —OR₁₂′ or —NR_{13′}R_{14′}, wherein R_{12′}, R_{13′}, and R_{14′}, stand for C₁-C₂₅-alkyl, C₆-C₁₂-cycloalkyl, C₆-C₂₄-aryl, or a saturated or unsaturated heterocyclic radical comprising five to seven ring atoms, wherein the ring consists of carbon atoms and one to three hetero atoms selected from the group consisting of nitrogen, oxygen and sulfur, wherein the aryl and heterocyclic radical can be substituted one to three times with C₁-C₈alkyl or C₁-C₈alkoxy, or —NR_{8′} R_{9′} stands for a five- or six-membered heterocyclic radical in which R_{8′} and R_{9′} together stand for tetramethylene, pentamethylene, —CH₂—CH₂—O—CH₂—CH₂—, or —CH₂—CH₂—NR₅—CH₂—CH₂—, preferably —CH₂—CH₂—O—CH₂—CH₂—, and n′ stands for 0, 1, 2 or 3. The DPP compounds can be used for the preparation of inks, colorants, pigmented plastics for coatings, non-impact-printing material, color filters, cosmetics, or for the preparation of polymeric ink particles, toners, dye lasers and electroluminescent devices.

EP-A-1087006 relates to an electroluminescent device comprising in this order (a) an anode, (b) a hole transporting layer, (c) a light-emitting layer, (d) optionally an electron transporting layer and (e) a cathode and a light-emitting substance, wherein the light-emitting substance is a diketopyrrolopyrrole (“DPP”) represented by formula 1′.

Further fluorescent DPP compounds and their use in electroluminescent devices are disclosed in EP 01810636.

Surprisingly, it was found that luminescent devices, which are high in the efficiency of electrical energy utilisation and high in luminance, can be obtained if specific combinations of DPP compounds are used as light emitting substances.

Accordingly, the present invention relates to compositions comprising a guest chromophore and a host chromophore, wherein the absorption spectrum of the guest chromophore overlaps with the fluorescence emission spectrum of the host chromophore, wherein the host chromophore is a diketopyrrolopyrrole having a photoluminescence emission peak at 500 to 720 nm, preferably 500 to 600 nm, most preferred 520 to 580 nm and wherein the guest chromophore is a diketopyrrolopyrrole having an absorption peak at 500 to 720 nm, preferably 500 to 600 nm, most preferred 520 to 580 nm

In a preferred embodiment, the present invention relates to compositions comprising a diketopyrrolopyrrole (“DPP”) represented by formula I 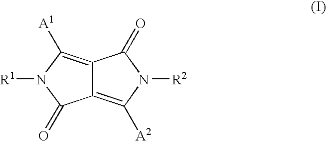
and a DPP represented by formula II 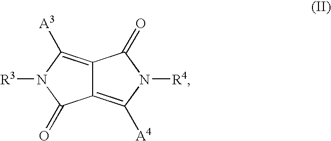
wherein R¹, R², R³ and R⁴ independently from each other stand for C₁-C₂₅-alkyl, which can be substituted by fluorine, chlorine or bromine, CS-C₁₋₂-cycloalkyl or C₅-C₁₂-cycloalkyl which can be condensed one or two times by phenyl which can be substituted one to three times with C₁-C₄-alkyl, halogen, nitro or cyano, silyl, A⁵ or —CR¹¹R¹²—(CH₂)ₘ-A⁵, wherein R¹¹ and R¹² independently from each other stand for hydrogen, fluorine, chlorine, bromine, cyano or C₁-C₄alkyl, which can be substituted by fluorine, chlorine or bromine, or phenyl which can be substituted one to three times with C₁-C₃alkyl, A⁵ stands for phenyl or 1- or 2-naphthyl which can be substituted one to three times with C₁-C₈alkyl, C₁-C₈alkoxy, halogen, nitro, cyano, phenyl, which can be substituted with C₁-C₈alkyl or C₁-C₈alkoxy one to three times, —NR¹³R¹⁴ wherein R¹³ and R¹⁴ represent hydrogen, C₁-C₂₅-alkyl, C₅-C₁₂-cycloalkyl or C₆-C₂₄-aryl, in particular phenyl or 1- or 2-naphthyl which can be substituted one to three times with C₁-C₈alkyl, C₁-C₈alkoxy, halogen or cyano, or phenyl, which can be substituted with C₁-C₈alkyl or C₁-C₈alkoxy one to three times, and m stands for 0, 1, 2, 3 or 4,
A¹ and A² independently from each other stand for 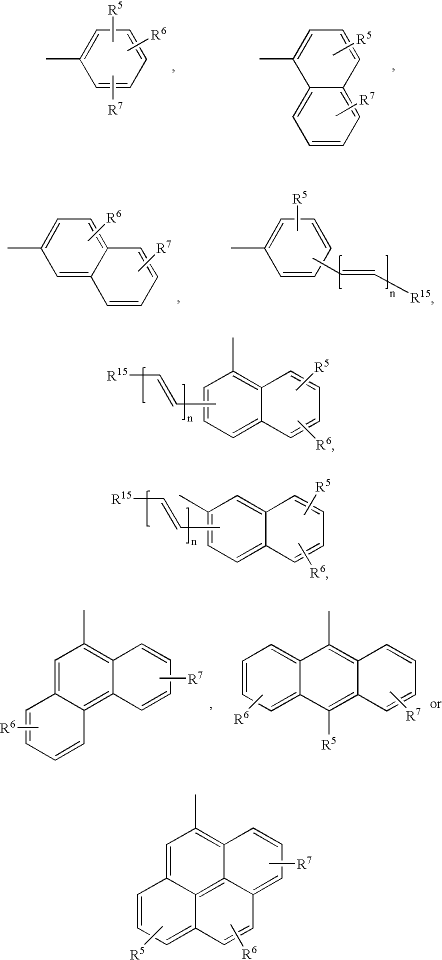
   wherein
R⁵, R⁶, R⁷ independently from each other stands for hydrogen, C₁-C₂₅-alkyl, C₁-C₂₅-alkoxy, —OCR¹¹R¹²—(CH₂)ₘ-A⁵, cyano, halogen, —OR¹⁰, —S(O)ₚR¹³, or phenyl, which can be substituted one to three times with C₁-C₈alkyl or C₁-C₈alkoxy, wherein R¹⁰ stands for C₆-C₂₄-aryl, or a saturated or unsaturated heterocyclic radical comprising five to seven ring atoms, wherein the ring consists of carbon atoms and one to three hetero atoms selected from the group consisting of nitrogen, oxygen and sulfur, R¹³ stands for C₁-C₂₅-alkyl, C₅-C₁₂-cycloalkyl, —CR¹¹R¹²—(CH₂)ₘ—Ph, R¹⁵ stands for C₆-C₂₄-aryl, p stands for 0, 1, 2 or 3 and n stands for 0, 1, 2, 3 or 4,
A³ and A⁴ independently from each other stand for 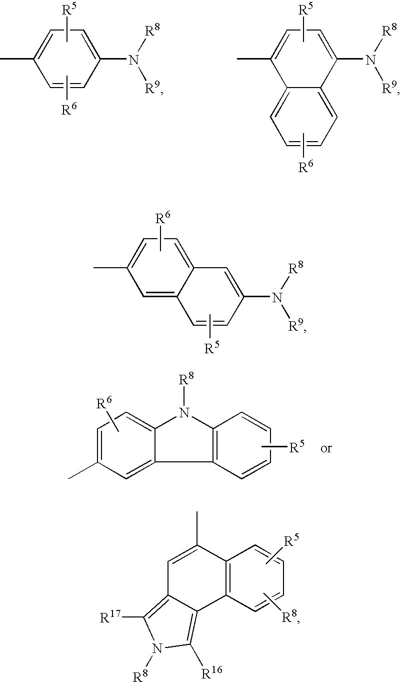
   wherein R⁸ and R⁹ independently from each other stand for hydrogen, C₁-C₂₅-alkyl, C₅-C₁₂-cycloalkyl, —CR¹¹R¹²_(CH₂)ₘ A⁵, C₆-C₂₄-aryl, in particular A¹, or a saturated or unsaturated heterocyclic radical comprising five to seven ring atoms, wherein the ring consists of carbon atoms and one to three hetero atoms selected from the group consisting of nitrogen, oxygen and sulfur, and R¹⁶ and R¹⁷ independently from each other stand for hydrogen and C₆-C₂₄-aryl, in particular phenyl; an electroluminescent device comprising the above-mentioned composition and the use of the composition for coloring a high molecular weight organic material, i.e. the use of the composition for the preparation of inks, colorants, pigmented plastics for coatings, non-impact-printing material, color filters, cosmetics, polymeric ink particles, toners, dye lasers and electroluminescent devices.

The present invention provides red or orange fluorescent compositions with a high heat stability, a good solubility in polymers, hydrocarbon based fuels, lubricants etc., a high light stability, and the ability to be used in plastics, especially polyamides, without decomposition and loss of lightfastness, and in paints and with a high electroluminescent (EL) emission intensity.

R¹, R², R³ and R⁴ independently from each other stand for C₁-C₂₅-alkyl, preferably C₁-C₈alkyl, in particular n-butyl, tert.-butyl and neopentyl, O₅—C₁₋₂cycloalkyl or C₅-C₁₂-cycloalkyl which can be condensed one or two times by phenyl which can be substituted one to three times with C₁-C₄-alkyl, halogen and cyano, in particular cyclohexyl, which can be substituted one to three times with C₁-C₈alkyl and/or C₁-C₈alkoxy, in particular 2,6-di-isopropylcyclohexyl, or 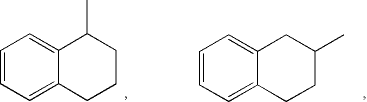
silyl, in particular trimethylsilyl, A⁵ or —CR¹¹R¹² (CH₂)ₘ-A⁵, wherein R¹¹ and R¹² independently from each other stand for hydrogen or C₁-C₄alkyl, or phenyl which can be substituted one to three times with C₁-C₃alkyl, A⁵ stands for phenyl or 1- or 2-naphthyl which can be substituted one to three times with C₁-C₈alkyl, C₁-C₈alkoxy, halogen, cyano, phenyl, which can be substituted with C₁-C₈alkyl or C₁-C₈alkoxy one to three times, or —NR¹³R¹⁴, wherein R¹³ and R¹⁴ represent C₁-C₂₅-alkyl, C₅-C₁₂-cycloalkyl or C₆-C₂₄-aryl, in particular phenyl or 1- or 2-naphthyl, which can be substituted one to three times with C₁-C₈alkyl, C₁-C₈alkoxy, halogen or cyano or phenyl, which can be substituted with C₁-C₈alkyl or C₁-C₈alkoxy one to three times, in particular 3,5-dimethylphenyl, 3,5-di-tert.-butylphenyl, 3-methylphenyl and 2,6-di-isopropylphenyl, and m stands for 0, 1, 2, 3 or 4, in particular 0 or 1.

Preferably R¹ and R² are independently of each other C₁-C₈alkyl, 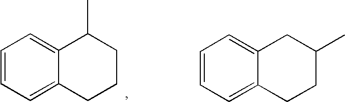
or —CR¹¹R¹²-A⁵, wherein R¹¹ is hydrogen, R¹² is hydrogen, in particular methyl or phenyl and A⁵ is 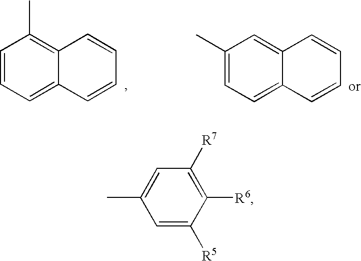
wherein R⁵, R⁶ and R⁷ are independently of each other hydrogen, C₁-C₄-alkyl, or halogen, in particular Br, wherein groups 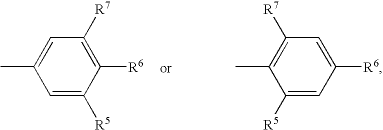
wherein R⁵, R⁶ and R⁷ are hydrogen; R⁶ is CO—C₄-alkyl, phenyl or Br and R⁵ and R⁷ are hydrogen; R⁵ is C₁-C₄-alkyl and R⁶ and R⁷ are hydrogen; or R⁶ is hydrogen and R⁵ and R⁷ are C₁-C₄-alkyl are most preferred.

Preferably R³ and R⁴ are independently of each other C₁-C₈-alkyl or —CR¹¹R¹²-A⁵, wherein R¹¹ is hydrogen, R¹² is methyl or phenyl, in particular hydrogen and A⁵ is 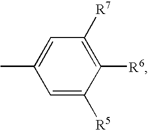
wherein R⁵, R⁶ and R⁷ are independently of each other hydrogen, C₁-C₄-alkyl, or CN, wherein groups 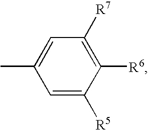
wherein R⁵, R⁶ and R⁷ are hydrogen; R⁶ is CN or C₁-C₄-alkyl and R⁵ and R⁷ are hydrogen, R⁵ and R⁶ are CN and R⁷ is hydrogen; R⁵ is C₁-C₄-alkyl and R⁶ and R⁷ are hydrogen; or R⁶ is hydrogen and R⁵ and R⁷ are C₁-C₄-alkyl are most preferred.

The weight ratio of the DPP compound of the formula I to the DPP compound of the formula II is in general 50:50 to 99.99:0.01, preferably 90:10 to 99.99:0.01, more preferably 95:5 to 99.9:0.1, most preferably 98:2 to 99.9:0.1.

The DPP compounds of the formula I and II are distinguished by the substituents A¹ and A² and A³ and A⁴, respectively.

A¹ and A² independently from each other stand for 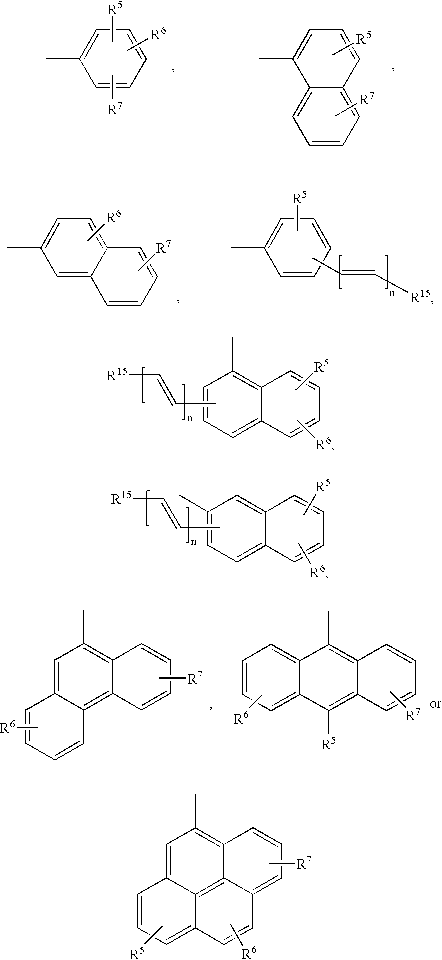
wherein R⁵, R⁶, R⁷, n and R¹⁵ have the above-mentioned meanings.

If the phenyl or naphthyl substituent is substituted by a vinyl group, A¹ and A² independently from each other can stand for 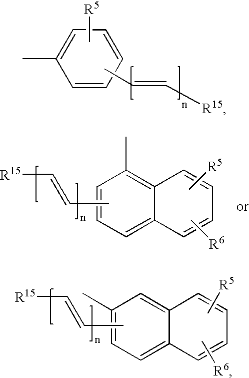
wherein n is an integer of 1 to 4, in particular 1 or 2, R⁵ and R⁶ independently from each other can stand for hydrogen, C₁-C₈alkyl or C₁-C₈alkoxy and R¹⁵ is C₆-C₂₄aryl, such as phenyl, 1-naphthyl, 2-naphthyl, 4-biphenyl, phenanthryl, terphenyl, pyrenyl, 2- or 9-fluorenyl or anthracenyl, preferably C₆-C₁₂aryl such as phenyl, 1-naphthyl, 2-naphthyl, 4-biphenyl, which may be unsubstituted or substituted by C₁-C₈alkyl or C₁-C₈alkoxy, wherein groups of the following formula are preferred:

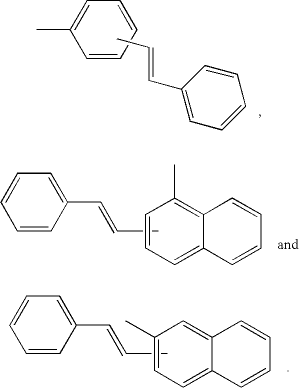

If A¹ and A² independently from each other stand for

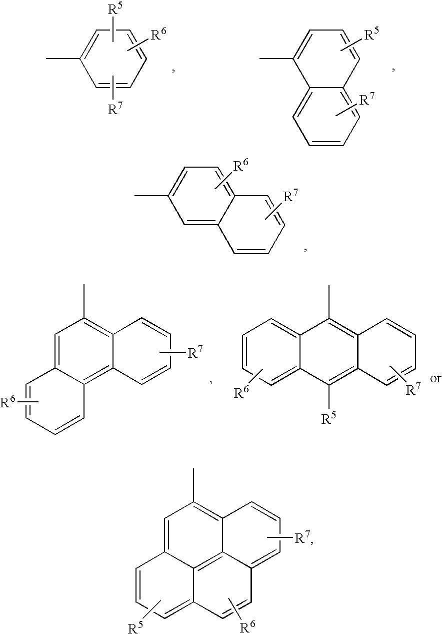

R⁵, R⁶ and R⁷ independently from each other stand for hydrogen, C₁-C₈-alkyl, C₁-C₈-alkoxy, —OCR¹¹R¹²—(CH₂)ₘ-A⁵, cyano, chloro, —OR¹⁰, or phenyl, which can be substituted one to three times with C₁-C₈alkyl or C₁-C₈alkoxy, wherein R¹⁰ stands for C₆-C₂₄-aryl, such as phenyl, 1-naphthyl or 2-naphthyl, R¹¹ and R¹² are hydrogen or C₁-C₄-alkyl, m is 0 or 1, A⁵ is phenyl, 1-naphthyl or 2-naphthyl, wherein groups of the following formula are preferred: 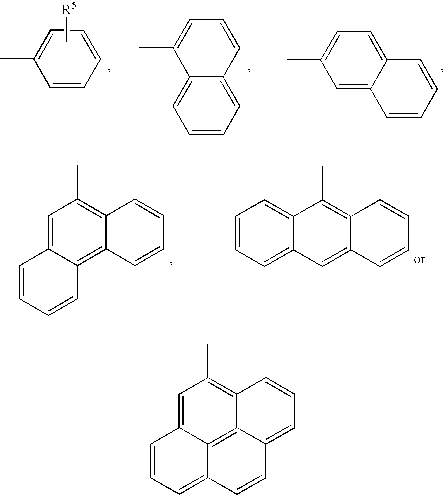
wherein R⁵ is C₁-C₈-alkyl.

In addition, DPP compounds of the formula I are preferred, wherein R¹ and R² are C₁-C₂₅-alkyl, in particular C₁-C₂₅-alkyl, wherein all or part of the hydrogen atoms are replaced by fluorine atoms, a group —CR¹¹R¹²-A⁵, wherein R¹¹ is hydrogen or C₁₋₄-alkyl, in particular methyl, R¹² is CF₃ or F, and A⁵ is phenyl, or a group —CR¹¹R¹²-A⁵, wherein R¹¹ is hydrogen, R¹² is C₁₋₄alkyl, in particular methyl, A⁵ is a group 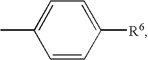
wherein R⁶ is fluorine, chlorine, bromine, preferably cyano or nitro.

The wording “C₁-C₂₅-alkyl, which are substituted by fluorine” comprises linear or branched C₁-C₂₅-alkyl groups wherein all or a part of the hydrogen atoms are replaced by fluorine atoms. Examples of such groups are —CH₂F, —CHF₂, —CF₃, FH₂CCH₂—, FH₂CCHF—, F₂HCCH₂—, F₂HCCHF—, F₃CCH₂—, F₂HCCF₂—, F₃CCHF—, F₃CCF₂—, CF₃CF₂CF₂—, CF₃CF₂CF₂CF₂—, or F₃C(CF₂)₃CF₂—,

Particularly preferred DPP compounds of the formula I are the following compounds:

| | |
|---|---|
| | (I) |
| 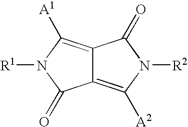 | |

| Compound | A¹ = A² | R¹ = R² |
|---|---|---|
| A-1 | 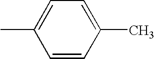 | 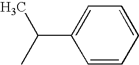 |
| A-2 | 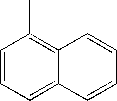 | 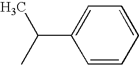 |
| A-3 | 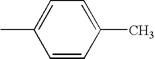 | 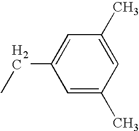 |
| A-4 | 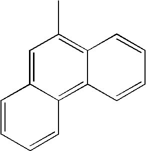 | 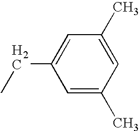 |
| A-5 | ″ | 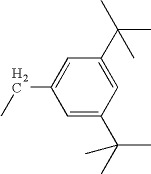 |
| A-6 | ″ | —(CH₂)₃CH₃ |
| A-7 | 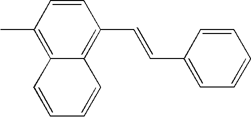 | 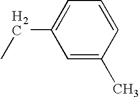 |
| A-8 | 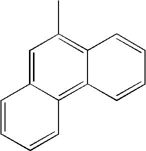 | —Si(CH₃)₃ |
| A-9 | 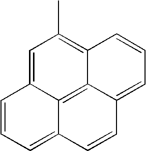 | 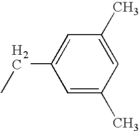 |
| A-10 | 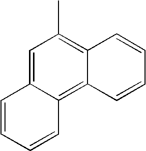 | 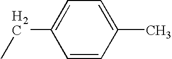 |
| A-11 | 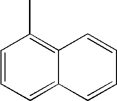 | 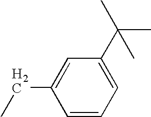 |
| A-12 | 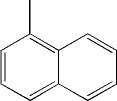 | 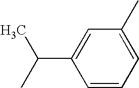 |
| A-13 | 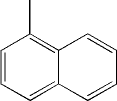 | 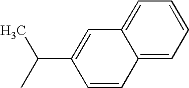 |
| A-14 | 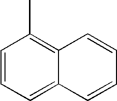 | 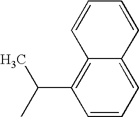 |
| A-15 | 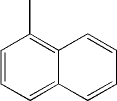 | 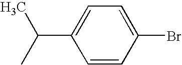 |
| A-16 | 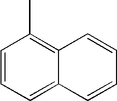 | 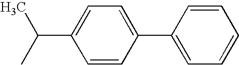 |
| A-17 | 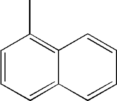 | —CH(CH₃)₂ |
| A-18 | 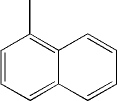 | 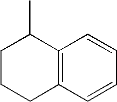 |
| A-19 | 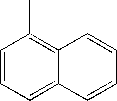 | 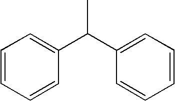 |
| A-20 | 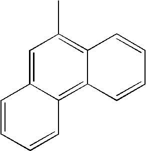 | 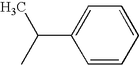 |
| A-21 | 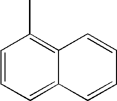 | 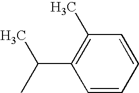 |
| A-22 | 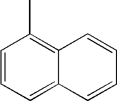 | 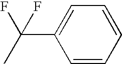 |
| A-23 | ″ | 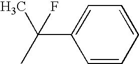 |
| A-24 | ″ | —CF₃ |
| A-25 | ″ | —CHF₂ |
| A-26 | ″ | —CH₂F |
| A-27 | ″ | 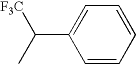 |
| A-28 | ″ | 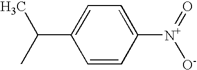 |
| A-29 | ″ | 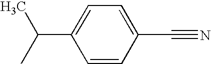 |

A³ and A⁴ independently from each other stand for 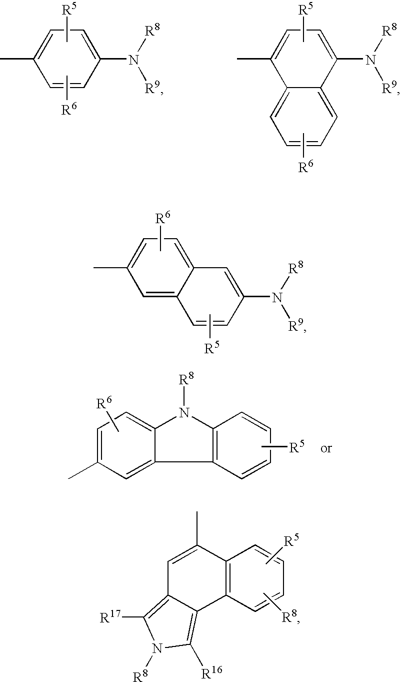
wherein R⁵, R⁶, R⁸, R⁹, R¹⁶ and R¹⁷ have the above-defined meanings.

If AP and A⁴ independently from each other stand for a group of the formula

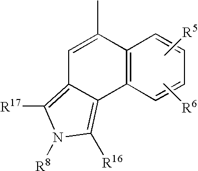

R⁵ and R⁶ are preferably hydrogen, R⁸ is preferably C₁-C₆alkyl or phenyl and R¹⁶ and R¹⁷ are preferably hydrogen or phenyl.

If A³ and A⁴ independently from each other stand for a group of the formula

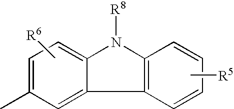

R⁵ and R⁶ are preferably hydrogen and R⁸ is preferably C₁-C₆alkyl or phenyl.

In particular A³ and A⁴ independently of each other stand for 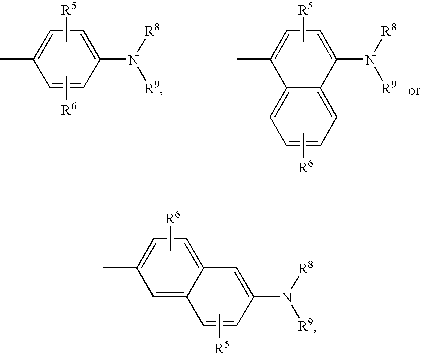
wherein R⁵, R⁶, R⁷ independently from each other stand for hydrogen, C₁-C₈-alkyl, C₁-C₈-alkoxy, —OCR¹¹R¹²_(CH₂)ₘ-A⁵, cyano, chloro, —OR¹⁰, or phenyl, which can be substituted one to three times with C₁-C₈alkyl or C₁-C₈alkoxy, wherein R¹⁰ stands for C₆-C₂₄-aryl, such as phenyl, 1-naphthyl or 2-naphthyl, R¹¹ and R¹² are hydrogen or C₁-C₄-alkyl, m is 0 or 1, A⁵ is phenyl, 1-naphthyl or 2-naphthyl, R⁸ and R⁹ independently from each other stand for hydrogen, C₁-C₈-alkyl, C₅-C₁₂-cycloalkyl, in particular cyclohexyl, —CR¹¹R¹²—(CH₂)ₘ-A⁵, C₆-C₂₄-aryl, such as phenyl, 1-naphthyl, 2-naphthyl, 4-biphenyl, phenanthryl, terphenyl, pyrenyl, 2- or 9-fluorenyl or anthracenyl, preferably C₆-C₁₂aryl such as phenyl, 1-naphthyl, 2-naphthyl, 4-biphenyl, which may be unsubstituted or substituted, in particular A¹, or a saturated or unsaturated heterocyclic radical comprising five to seven ring atoms, wherein the ring consists of carbon atoms and one to three hetero atoms selected from the group consisting of nitrogen, oxygen and sulfur.

In particular groups of the following formula are preferred 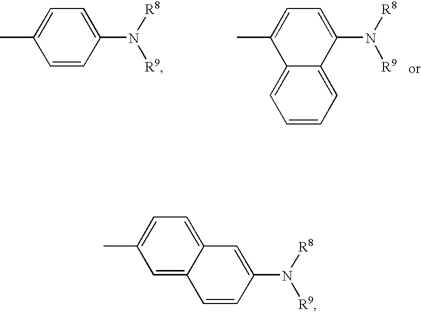
wherein R⁸ and R⁹ are independently of each other a group of the formula

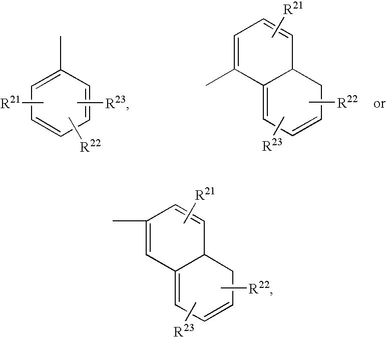

wherein R²′, R²² and R²³ are independently of each other hydrogen, C₁-C₈alkyl, a hydroxyl group, a mercapto group, C₁-C₈alkoxy, C₁-C₈alkylthio, halogen, halo-C₁-C₈alkyl, a cyano group, an aldehyde group, a ketone group, a carboxyl group, an ester group, a carbamoyl group, an amino group, a nitro group, a silyl group or a siloxanyl group. Preferably R²¹, R²² and R²³ are independently of each other hydrogen, C₁-C₈alkyl, C₁-C₈alkoxy or C₁-C₈alkylthio. Particularly preferred DPP compounds of the formula II are the following compounds:

| | |
|---|---|
| | (II) |
| 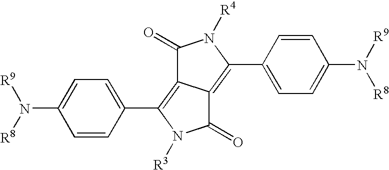 | |

| Compound | R³ = R⁴ | R⁸ | R⁹ |
|---|---|---|---|
| B-1 | 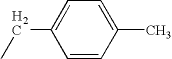 | 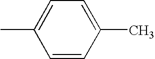 | 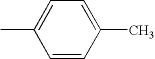 |
| B-2 | —(CH₂)₃CH₃ | 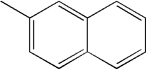 | 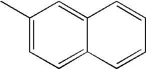 |
| B-3 | 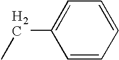 | 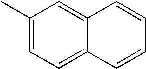 | 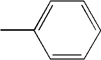 |
| B-4 | 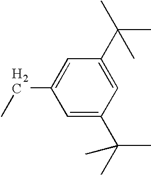 | 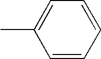 | 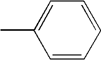 |
| B-5 | 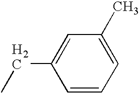 | ″ | ″ |
| B-6 | ″ | 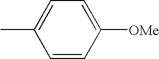 | 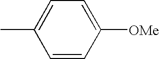 |
| B-7 | 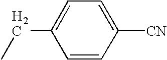 | 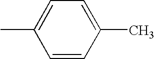 | 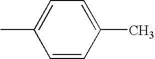 |
| B-8 | 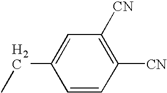 | 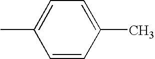 | 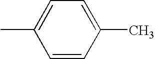 |
| B-9 | 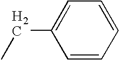 | 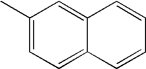 | 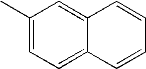 |

Particularly preferred inventive compositions comprise compounds A-2 and B-1, A-2 and B-3, A-2 and B-7, A-11 and B-1 or A-11 and B-7.

The inventive DPP compounds of formula I or II can be synthesized according to or in analogy to methods well known in the art, such as described, for example, in U.S. Pat. No. 4,579,949, EP-A 353,184, EP-A-133,156, EP-A-1,087,005 and EP-A-1,087,006.

The term “halogen” means fluorine, chlorine, bromine and iodine.

C₁-C₂₅alkyl is typically linear or branched—where possible—methyl, ethyl, n-propyl, isopropyl, n-butyl, sec.-butyl, isobutyl, tert.-butyl, n-pentyl, 2-pentyl, 3-pentyl, 2,2-dimethylpropyl, n-hexyl, n-heptyl, n-octyl, 1,1,3,3-tetramethylbutyl and 2-ethylhexyl, n-nonyl, decyl, undecyl, dodecyl, tetradecyl, pentadecyl, hexadecyl, heptadecyl, octadecyl, eicosyl, heneicosyl, docosyl, tetracosyl or pentacosyl, preferably C₁-C₈alkyl such as methyl, ethyl, n-propyl, isopropyl, n-butyl, sec.-butyl, isobutyl, tert.-butyl, n-pentyl, 2-pentyl, 3-pentyl, 2,2-dimethyl-propyl, n-hexyl, n-heptyl, n-octyl, 1,1,3,3-tetramethylbutyl and 2-ethylhexyl, more preferably C₁-C₄alkyl such as typically methyl, ethyl, n-propyl, isopropyl, n-butyl, sec.-butyl, isobutyl, tert.-butyl; C₁-C₃alkyl stands for methyl, ethyl, n-propyl, or isopropyl; C₁-C₆alkyl stands for methyl, ethyl, n-propyl, isopropyl, n-butyl, sec.-butyl, isobutyl, tert.-butyl, n-pentyl, 2-pentyl, 3-pentyl, 2,2-dimethyl-propyl, or n-hexyl.

The “aldehyde group, ketone group, ester group, carbamoyl group and amino group” include those substituted by an aliphatic hydrocarbon group, an alicyclic hydrocarbon group, an aromatic hydrocarbon group, a heterocyclic group or the like, wherein the aliphatic hydrocarbon group, alicyclic hydrocarbon group, aromatic hydrocarbon group and heterocyclic group may be unsubstituted or substituted. The term “silyl group” means a silicon compound group such as trimethylsilyl. The term “siloxanyl group” means a silicon compound group linking through intermediation of an ether linkage, such as trimethylsiloxanyl and the like.

Examples of C₁-C₈alkoxy are methoxy, ethoxy, n-propoxy, isopropoxy, n-butoxy, sec.-butoxy, isobutoxy, tert.-butoxy, n-pentoxy, 2-pentoxy, 3-pentoxy, 2,2-dimethylpropoxy, n-hexoxy, n-heptoxy, n-octoxy, 1,1,3,3-tetramethylbutoxy and 2-ethylhexoxy, preferably C₁-C₄alkoxy such as typically methoxy, ethoxy, n-propoxy, isopropoxy, n-butoxy, sec.-butoxy, isobutoxy, tert.-butoxy. The term “alkylthio group” means the same groups as the alkoxy groups, except that the oxygen atom of ether linkage is replaced by a sulfur atom.

The term “aryl group” is typically C₆-C₂₄aryl, such as phenyl, 1-naphthyl, 2-naphthyl, 4-biphenyl, phenanthryl, terphenyl, pyrenyl, 2- or 9-fluorenyl or anthracenyl, preferably C₆-C₁₂aryl such as phenyl, 1-naphthyl, 2-naphthyl, 4-biphenyl, which may be unsubstituted or substituted.

The term “cycloalkyl group” is typically C₅-C₁₂cycloalkyl, such as cyclopentyl, cyclohexyl, cycloheptyl, cyclooctyl, cyclononyl, cyclodecyl, cycloundecyl, cyclododecyl, preferably cyclopentyl, cyclohexyl, cycloheptyl, or cyclooctyl, may be unsubstituted or substituted. The term “cycloalkenyl group” means an unsaturated alicyclic hydrocarbon group containing one or more double bonds, such as cyclopentenyl, cyclopentadienyl, cyclohexenyl and the like, which may be unsubstituted or substituted. The cycloalkyl group, in particular a cyclohexyl group, can be condensed one or two times by phenyl which can be substituted one to three times with C₁-C₄-alkyl, halogen and cyano. Examples of such condensed cyclohexyl groups are: 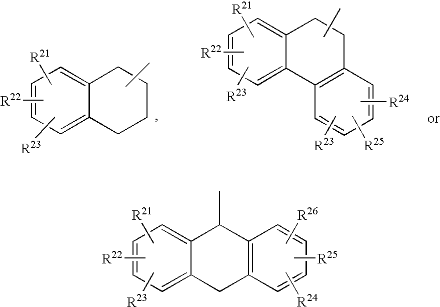
in particular 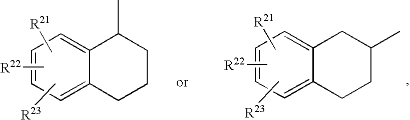
wherein R²¹, R²²R²³, R²⁴, R²⁵ and R²⁶ are independently of each other C₁-C₄-alkyl, halogen and cyano, in particular hydrogen. The term “heterocyclic radical” is a ring with five to seven ring atoms, wherein nitrogen, oxygen or sulfur are the possible hetero atoms, and is typically an unsaturated heterocyclic radical with five to 18 atoms having at least six conjugated π-electrons such as thienyl, benzo[b]thienyl, dibenzo[b,d]thienyl, thianthrenyl, furyl, furfuryl, 2H-pyranyl, benzofuranyl, isobenzofuranyl, dibenzofuranyl, phenoxythienyl, pyrrolyl, imidazolyl, pyrazolyl, pyridyl, bipyridyl, triazinyl, pyrimidinyl, pyrazinyl, pyridazinyl, indolizinyl, isoindolyl, indolyl, indazolyl, purinyl, quinolizinyl, chinolyl, isochinolyl, phthalazinyl, naphthyridinyl, chinoxalinyl, chinazolinyl, cinnolinyl, pteridinyl, carbazolyl, carbolinyl, benzotriazolyl, benzoxazolyl, phenanthridinyl, acridinyl, perimidinyl, phenanthrolinyl, phenazinyl, isothiazolyl, phenothiazinyl, isoxazolyl, furazanyl or phenoxazinyl, preferably the above-mentioned mono- or bicyclic heterocyclic radicals.

The above-mentioned substituents can be substituted by a C₁-C₈alkyl, a hydroxyl group, a mercapto group, C₁-C₈alkoxy, C₁-C₈alkylthio, halogen, halo-C₁-C₈alkyl, a cyano group, an aldehyde group, a ketone group, a carboxyl group, an ester group, a carbamoyl group, an amino group, a nitro group, a silyl group or a siloxanyl group,

The present invention relates further to an electroluminescent device having the composition according to the present invention between an anode and a cathode and emitting light by the action of electrical energy.

Typical constitutions of latest organic electroluminescent devices are:
(i) an anode/a hole transporting layer/an electron transporting layer/a cathode, in which the compositions are used either as positive-hole transport composition, which is exploited to form the light emitting and hole transporting layers, or as electron transport compositions, which can be exploited to form the light-emitting and electron transporting layers, and
(ii) an anode/a hole transporting layer/a light-emitting layer/an electron transporting layer/a cathode, in which the compositions form the light-emitting layer regardless of whether they exhibit positive-hole or electron transport properties in this constitution, and
(iii) an anode/a hole injection layer/a hole transporting layer/a light-emitting layer/an electron transporting layer/a cathode, and
(iv) an anode/a hole transporting layer/a light-emitting layer/a positive hole inhibiting layer/an electron transporting layer/a cathode, and
(v) an anode/a hole injection layer/a hole transporting layer/a light-emitting layer/a positive hole inhibiting layer/an electron transporting layer/a cathode.

Thin film type electroluminescent devices usually consist essentially of a pair of electrodes and at least one charge transporting layer in between. Usually two charge transporting layers, a hole transporting layer (next to the anode) and an electron transporting layer (next to the cathode) are present. Either one of them contains—depending on its properties as hole-transporting or electron-transporting material—an inorganic or organic fluorescence substance as light-emitting material. It is also common, that a light-emitting material is used as an additional layer between the hole-transporting and the electron-transporting layer. In the above mentioned device structure, a hole injection layer can be constructed between a anode and a hole transporting layer and/or a positive hole inhibiting layer can be constructed between a light emitting layer and a electron transporting layer to maximise hole and electron population in the light emitting layer, reaching large efficiency in charge recombination and intensive light emission.

The devices can be prepared in several ways. Usually, vacuum evaporation is used for the preparation. Preferably, the organic layers are laminated in the above order on a commercially available indium-tin-oxide (“ITO”) glass substrate held at room temperature, which works as the anode in the above constitutions. The membrane thickness is preferably in the range of 1 to 10,000 nm, more preferably 1 to 5,000 nm, more preferably 1 to 1,000 nm, more preferably 1 to 500 nm. The cathode metal, such as a Mg/Ag alloy or a binary Li—Al system of ca. 200 nm is laminated on the top of the organic layers. The vacuum during the deposition is preferably less than 0.1333 Pa (1×10⁻³ Torr), more preferably less than 1.333×10⁻³ Pa (1×10⁻⁵ Torr), more preferably less than 1.333×10⁻⁴ Pa (1×10⁻⁶ Torr).

As anode usual anode materials which possess high work function such as metals like gold, silver, copper, aluminum, indium, iron, zinc, tin, chromium, titanium, vanadium, cobalt, nickel, lead, manganese, tungsten and the like, metallic alloys such as magnesium/copper, magnesium/silver, magnesium/aluminum, aluminum/indium and the like, semiconductors such as Si, Ge, GaAs and the like, metallic oxides such as indium-tin-oxide (“ITO”), ZnO and the like, metallic compounds such as CuI and the like, and furthermore, electroconducting polymers such polyacetylene, polyaniline, polythiophene, polypyrrole, polyparaphenylene and the like, preferably ITO, most preferably ITO on glass as substrate can be used.

Of these electrode materials, metals, metallic alloys, metallic oxides and metallic compounds can be transformed into electrodes, for example, by means of the sputtering method. In the case of using a metal or a metallic alloy as a material for an electrode, the electrode can be formed also by the vacuum deposition method. In the case of using a metal or a metallic alloy as a material forming an electrode, the electrode can be formed, furthermore, by the chemical plating method (see for example, Handbook of Electrochemistry, pp 383-387, Mazuren, 1985). In the case of using an electroconducting polymer, an electrode can be made by forming it into a film by means of anodic oxidation polymerization method onto a substrate which is previously provided with an electroconducting coating. The thickness of an electrode to be formed on a substrate is not limited to a particular value, but, when the substrate is used as a light emitting plane, the thickness of the electrode is preferably within the range of from 1 nm to 100 nm, more preferably, within the range of from 5 to 50 nm so as to ensure transparency.

In a preferred embodiment ITO is used on a substrate having an ITO film thickness in the range of from 10 nm (100 Å) to 1μ (10000 Å), preferably from 20 nm (200 Å) to 500 nm (5000 Å). Generally, the sheet resistance of the ITO film is chosen in the range of not more than 100 Ω/cm², preferably not more than 50 Ω/cm².

Such anodes are commercially available from Japanese manufacturers, such as Geomatech Co. Ltd., Sanyo Vacuum Co. Ltd., Nippon Sheet Glass Co. Ltd.

As substrate either an electronconducting or electrically insulating material can be used. In case of using an electroconducting substrate, a light emitting layer or a positive hole transporting layer is directly formed thereupon, while in case of using an electrically insulating substrate, an electrode is firstly formed thereupon and then a light emitting layer or a positive hole transporting layer is superposed.

The substrate may be either transparent, semi-transparent or opaque. However, in case of using a substrate as an indicating plane, the substrate must be transparent or semi-transparent.

Transparent electrically insulating substrates are, for example, inorganic compounds such as glass, quartz and the like, organic polymeric compounds such as polyethylene, polypropylene, polymethylmethacrylate, polyacrylonitrile, polyester, polycarbonate, polyvinylchloride, polyvinylalcohol, polyvinylacetate and the like. Each of these substrates can be transformed into a transparent electroconducting substrate by providing it with an electrode according to one of the methods described above.

Examples of semi-transparent electrically insulating substrates are inorganic compounds such as alumina, YSZ (yttrium stabilized zirconia) and the like, organic polymeric compounds such as polyethylene, polypropylene, polystyrene, epoxy resins and the like. Each of these substrates can be transformed into a semi-transparent electroconducting substrate by providing it with an electrode according to one of the abovementioned methods.

Examples of opaque electroconducting substrates are metals such as aluminum, indium, iron, nickel, zinc, tin, chromium, titanium, copper, silver, gold, platinum and the like, various elctroplated metals, metallic alloys such as bronze, stainless steel and the like, semiconductors such as Si, Ge, GaAs, and the like, electroconducting polymers such as polyaniline, polythiophene, polypyrrole, polyacetylene, polyparaphenylene and the like.

A substrate can be obtained by forming one of the above listed substrate materials to a desired dimension. It is preferred that the substrate has a smooth surface. Even if it has a rough surface, it will not cause any problem for practical use, provided that it has round unevenness having a curvature of not less than 20 μm. As for the thickness of the substrate, there is no restriction as far as it ensures sufficient mechanical strength. As cathode usual cathode materials which possess low work function such as alkali metals, earth alkaline metals, group 13 elements, silver, and copper as well as alloys or mixtures thereof such as sodium, lithium, potassium, sodium-potassium alloy, magnesium, magnesium-silver alloy, magnesium-copper alloy, magnesium-aluminum alloy, magnesium-indium alloy, aluminum, aluminum-aluminum oxide alloy, aluminum-lithium alloy, indium, calcium, and materials exemplified in EP-A 499,011 such as electroconducting polymers e.g. polypyrrole, polythiophene, polyaniline, polyacetylene etc., preferably Mg/Ag alloys, or Li—Al compositions can be used.

In a preferred embodiment a magnesium-silver alloy or a mixture of magnesium and silver, or a lithium-aluminum alloy or a mixture of lithium and aluminum can be used in a film thickness in the range of from 10 nm (100 Å) to 1 μm (10000 Å), preferably from 20 nm (200 Å) to 500 nm (5000 Å).

Such cathodes can be deposited on the foregoing electron transporting layer by known vacuum deposition techniques described above.

In a preferred ambodiment of this invention a light-emitting layer can be used between the hole transporting layer and the electron transporting layer. Usually the light-emitting layer is prepared by forming a thin film on the hole transporting layer.

As methods for forming said thin film, there are, for example, the vacuum deposition method, the spin-coating method, the casting method, the Langmuir-Blodgett (“LB”) method and the like. Among these methods, the vacuum deposition method, the spin-coating method and the casting method are particularly preferred in view of ease of operation and cost.

In case of forming a thin film using a composition by means of the vacuum deposition method, the conditions under which the vacuum deposition is carried out are usually strongly dependent on the properties, shape and crystalline state of the compound(s). However, optimum conditions are usually as follows: temperature of the heating boat: 100 to 400° C.; substrate temperature: −100 to 350° C.; pressure: 1.33×10⁴ Pa (1×10² Torr) to 1.33×10⁻⁴ Pa (1×10⁻⁶ Torr) and deposition rate: 1 pm to 6 nm/sec.

In an organic EL element, the thickness of the light emitting layer is one of the factors determining its light emission properties. For example, if a light emitting layer is not sufficiently thick, a short circuit can occur quite easily between two electrodes sandwiching said light emitting layer, and therefor, no EL emission is obtained. On the other hand, if the light emitting layer is excessively thick, a large potential drop occurs inside the light emitting layer because of its high electrical resistance, so that the threshold voltage for EL emission increases. Accordingly, the thickness of the organic light emitting layer is limited to the range of from 5 nm to 5 μm, preferably to the range of from 10 nm to 500 nm.

In the case of forming a light emitting layer by using the spin-coating method and the casting method, the coating can be carried out using a solution prepared by dissolving the composition in a concentration of from 0.0001 to 90% by weight in an appropriate organic solvent such as benzene, toluene, xylene, tetrahydrofurane, methyltetrahydrofurane, N,N-dimethylformamide, dichloromethane, dimethylsulfoxide and the like. If the concentration exceeds 90% by weight, the solution usually is so viscous that it no longer permits forming a smooth and homogenous film. On the other hand, if the concentration is less than 0.0001% by weight, the efficiency of forming a film is too low to be economical. Accordingly, a preferred concentration of the composition is within the range of from 0.01 to 80% by weight. In the case of using the above spin-coating or casting method, it is possible to further improve the homogeneity and mechanical strength of the resulting layer by adding a polymer binder to the solution for forming the light emitting layer. In principle, any polymer binder may be used, provided that it is soluble in the solvent in which the composition is dissolved. Examples of such polymer binders are polycarbonate, polyvinylalcohol, polymethacrylate, polymethylmethacrylate, polyester, polyvinylacetate, epoxy resin and the like. However, if the solid content composed of the polymer binder and the composition exceeds 99% by weight, the fluidity of the solution is usually so low that it is impossible to form a light emitting layer excellent in homogeneity. On the other hand, if the content of the composition is substantially smaller than that of the polymer binder, the electrical resistance of said layer is very large, so that it does not emit light unless a high voltage is applied thereto. Accordingly, the preferred ratio of the polymer binder to the composition is chosen within the range of from 10:1 to 1:50 by weight, and the solid content composed of both components in the solution is preferably within the range of from 0.01 to 80% by weight, and more preferably, within the range of 0.1 to 60% by weight.

As hole-transporting layers known organic hole transporting compounds such as polyvinyl carbazole 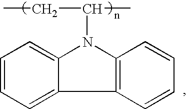
a TPD compound disclosed in J. Amer. Chem. Soc. 90 (1968) 3925: 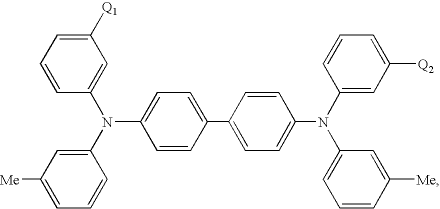
wherein Q₁ and Q₂ each represent a hydrogen atom or a methyl group; a compound disclosed in J. Appl. Phys. 65(9) (1989) 3610: 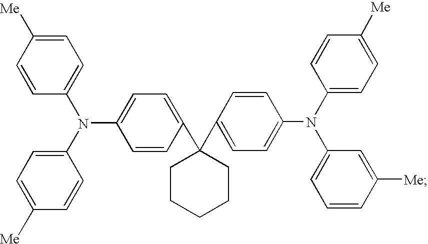
a stilbene based compound 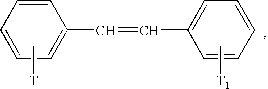
wherein T and T₁ stand for an organic radical;
a hydrazone based compound 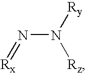
   wherein Rx, Ry and Rz stand for an organic radical, and the like can be used.

Compounds to be used as a positive hole transporting material are not restricted to the above listed compounds. Any compound having a property of transporting positive holes can be used as a positive hole transporting material such as-triazole derivatives, oxadiazole derivatives, imidazole derivatives, polyarylalkane derivatives, pyrazoline derivative, pyrazolone derivatives, phenylene diamine derivatives, arylamine derivatives, amino substituted chalcone derivatives, oxazole derivatives, stilbenylanthracene derivatives, fluorenone derivatives, hydrazone derivatives, stilbene derivatives, copolymers of aniline derivatives, electro-conductive oligomers, particularly thiophene oligomers, porphyrin compounds, aromatic tertiary amine compounds, stilbenyl amine compounds etc. Particularly, aromatic tertiary amine compounds such as N,N,N′,N′-tetraphenyl-4,4′-diaminobiphenyl, N,N′-diphenyl-N,N′-bis(3-methylphenyl)-4,4′-diaminobiphenyl (TPD), 2,2′-bis(di-p-torylaminophenyl)propane, 1,1′-bis(4-di-torylaminophenyl)-4-phenylcyclohexane, bis(4-dimethylamino-2-methylphenyl)phenylmethane, bis(4-di-p-tolylaminophenyl)phenyl-methane, N,N′-diphenyl-N,N′-di(4-methoxyphenyl)-4,4′-diaminobiphenyl, N,N,N′,N′-tetraphenyl-4,4′-diaminodiphenylether, 4,4′-bis(diphenylamino)quaterphenyl, N,N,N-tri(p-tolyl)amine, 4-(di-p-tolylamino)-4′-[4-(di-p-tolylamino)stilyl]stilbene, 4-N,N-diphenylamino-(2-diphenylvinyl)benzene, 3-methoxy-4′-N,N-diphenylaminostilbene, N-phenylcarbazole etc. are used.

Furthermore, 4,4′-bis[N-(1-naphtyl)-N-phenylamino]biphenyl disclosed in U.S. Pat. No. 5,061,569 and the compounds disclosed in EP-A 508,562, in which three triphenylamine units are bound to a nitrogen atom, such as 4,4′,4″-tris[N-(3-methylphenyl)-N-phenylamino]triphenylamine, can be used.

A positive hole transporting layer can be formed by preparing an organic film containing at least one positive hole transporting material on the anode. The positive hole transporting layer can be formed by the vacuum deposition method, the spin-coating method, the casting method, the LB method and the like. Of these methods, the vacuum deposition method, the spin-coating method and the casting method are particularly preferred in view of ease and cost.

In the case of using the vacuum deposition method, the conditions for deposition may be chosen in the same manner as described for the formation of a light emitting layer (see above). If it is desired to form a positive hole transporting layer comprising more than one positive hole transporting material, the coevaporation method can be employed using the desired compounds.

In the case of forming a positive hole transporting layer by the spin-coating method or the casting method, the layer can be formed under the conditions described for the formation of the light emitting layer (see above).

As in the case of forming the light emitting layer a smoother and more homogeneous positive hole transporting layer can be formed by using a solution containing a binder and at least one positive hole transporting material. The coating using such a solution can be performed in the same manner as described for the light emitting layer. Any polymer binder may be used, provided that it is soluble in the solvent in which the at least one positive hole transporting material is dissolved. Examples of appropriate polymer binders and of appropriate and preferred concentrations are given above when describing the formation of a light emitting layer.

The thickness of the positive hole transporting layer is preferably chosen in the range of from 0.5 to 1000 nm, preferably from 1 to 100 nm, more preferably from 2 to 50 nm.

As hole injection materials known organic hole transporting compounds such as metal-free phthalocyanine (H₂Pc), copper-phthalocyanine (Cu—Pc) and their derivatives as described, for example, in JP64-7635 can be used. Furthermore, some of the aromatic amines defined as hole transporting materials above, which have a lower ionisation potential than the hole transporting layer, can be used.

A hole injection layer can be formed by preparing an organic film containing at least one hole injection material between the anode layer and hole transporting layer. The hole injection layer can be formed by the vacuum deposition method, the spin-coating method, the casting method, the LB method and the like. The thickness of the layer is preferably from 5 nm to 5 μm, and more preferably from 10 nm to 100 nm.

The electron transporting materials should have a high electron injection efficiency (from the cathode) and a high electron mobility. The following materials can be exemplified for electron transporting materials: tris(8-hydroxyquinolinato)-aluminum(III) and its derivatives, bis(10-hydroxybenzo[h]quinolinolato)beryllium(II) and its derivatives, oxadiazole derivatives, such as 2-(4-biphenyl)-5-(4-tert.-butylphenyl)-1,3,4-oxadiazole and its dimer systems, such as 1,3-bis(4-tert.-butylphenyl-1,3,4)oxadiazolyl)biphenylene and 1,3-bis(4-tert.-butylphenyl-1,3,4-oxadiazolyl)phenylene, dioxazole derivatives, triazole derivatives, coumarine derivatives, imidazopyridine derivatives, phenanthroline derivatives or perylene tetracarboxylic acid derivatives disclosed in Appl. Phys. Lett. 48 (2) (1986) 183.

An electron transporting layer can be formed by preparing an organic film containing at least one electron transporting material on the hole transporting layer or on the light-emitting layer. The electron transporting layer can be formed by the vacuum deposition method, the spin-coating method, the casting method, the LB method and the like.

It is preferred that the positive hole inhibiting materials for a positive hole inhibiting layer have high electron injection/transporting efficiency from the electron transporting layer to the light emission layer and also have higher ionisation potential than the light emitting layer to prevent the flowing out of positive holes from the light emitting layer to avoid a drop in luminescence efficiency.

As the positive hole inhibiting material known materials, such as Balq, TAZ and phenanthroline derivatives, e.g. bathocuproine (BCP), can be used:

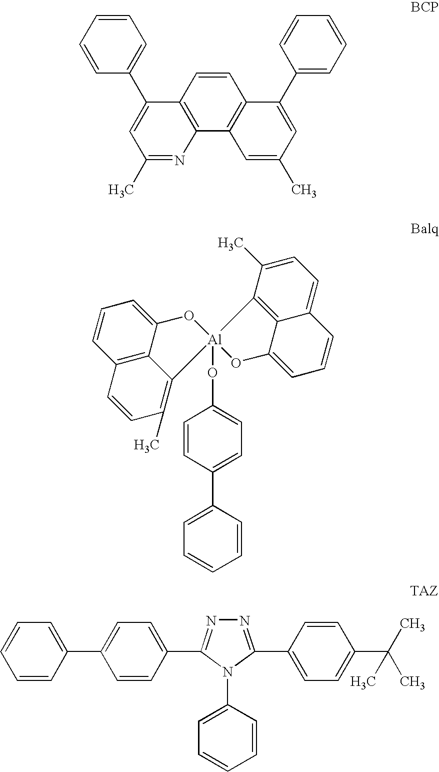

The positive hole inhibiting layer can be formed by preparing an organic film containing at least one positive hole inhibiting material between the electron transporting layer and the light-emitting layer. The positive hole inhibiting layer can be formed by the vacuum deposition method, the spin-coating method, the casting method, the LB method and the like. The thickness of the layer preferably is chosen within the range of from 5 nm to 2 μM, and more preferably, within the range of from 10 nm to 100 nm.

As in the case of forming a light emitting layer or a positive hole transporting layer, a smoother and more homogeneous electron transporting layer can be formed by using a solution containing a binder and at least one electron transporting material.

The thickness of an electron transporting layer is preferably chosen in the range of from 0.5 to 1000 nm, preferably from 1 to 100 nm, more preferably from 2 to 50 nm.

The light-emitting compositions have a fluorescence emission maximum in the range of from 500 to 780, preferably from 520 to 750, more preferred from 540 to 700 nm. Further, the inventive compounds preferably exhibit an absorption maximum in the range of 450 to 580 nm.

The light-emitting compositions usually exhibit a fluorescence quantum yield (“FQY”) in the range of from 1>FQY≧0.3 (measured in aerated toluene or DMF). Further, in general, the inventive compositions exhibit a molar absorption coefficient in the range of from 5000 to 100000.

Another embodiment of the present invention relates to a method of coloring high molecular weight organic materials (having a molecular weight usually in the range of from 10³ to 10⁷ g/mol; comprising biopolymers, and plastic materials, including fibres) by incorporating therein the inventive composition by methods known in the art.

The inventive compositions can be used, as described for the DPP compounds of formula I′ in EP-A-1087005, for the preparation of
inks, for printing inks in printing processes, for flexographic printing, screen printing, packaging printing, security ink printing, intaglio printing or offset printing, for pre-press stages and for textile printing, for office, home applications or graphics applications, such as for paper goods, for example, for ballpoint pens, felt tips, fiber tips, card, wood, (wood) stains, metal, inking pads or inks for impact printing processes (with impact-pressure ink ribbons), for the preparation of
colorants, for coating materials, for industrial or commercial use, for textile decoration and industrial marking, for roller coatings or powder coatings or for automotive finishes, for high-solids (low-solvent), water-containing or metallic coating materials or for pigmented formulations for aqueous paints, for the preparation of
pigmented plastics for coatings, fibers, platters or mold carriers, for the preparation of
non-impact-printing material for digital printing, for the thermal wax transfer printing process, the ink jet printing process or for the thermal transfer printing process, and also for the preparation of
color filters, especially for visible light in the range from 400 to 700 nm, for liquid-crystal displays (LCDs) or charge combined devices (CCDs) or for the preparation of
cosmetics or for the preparation of
polymeric ink particles, toners, dye lasers, dry copy toners liquid copy toners, or electrophotographic toners, and electroluminescent devices.

Another preferred embodiment concerns the use of the inventive compositions for color changing media. There are three major techniques in order to realize full-color organic electroluminescent devices:
(i) use of the three primary colors blue, green and red generated by electroluminescence,
(ii) conversion of the electroluminescent blue or white to photoluminescent green and red via color changing media (CCM), which absorb the above electroluminescent blue, and fluorescence in green and red.
(iii) conversion of the white luminescent emission to blue, green and red via color filters.

The inventive compounds are useful for EL materials for the above category (i) and, in addition, for the above mention technique (ii). This is because the invented combinations of compounds can exhibit strong photoluminescence as well as electrolunimescence.

Technique (ii) is, for example, known from U.S. Pat. No. 5,126,214, wherein EL blue with a maximum wavelength of ca. 470-480 nm is converted to green and red using coumarin, 4-(dicyanomethylene)-2-methyl-6-(p-dimethylaminostyryl)-4H-pyran, pyridine, rhodamine 6G, phenoxazone or other dyes.

Illustrative examples of suitable organic materials of high molecular weight which can be colored with the inventive compositions are described in EP-A-1087005.

Particularly preferred high molecular weight organic materials, in particular for the preparation of a paint system, a printing ink or ink, are, for example, cellulose ethers and esters, e.g. ethylcellulose, nitrocellulose, cellulose acetate and cellulose butyrate, natural resins or synthetic resins (polymerization or condensation resins) such as aminoplasts, in particular urea/formaldehyde and melamine/formaldehyde resins, alkyd resins, phenolic plastics, polycarbonates, polyolefins, polystyrene, polyvinyl chloride, polyamides, polyurethanes, polyester, ABS, ASA, polyphenylene oxides, vulcanized rubber, casein, silicone and silicone resins as well as their possible mixtures with one another.

It is also possible to use high molecular weight organic materials in dissolved form as film formers, for example boiled linseed oil, nitrocellulose, alkyd resins, phenolic resins, melamine/formaldehyde and urea/formaldehyde resins as well as acrylic resins.

Said high molecular weight organic materials may be obtained singly or in admixture, for example in the form of granules, plastic materials, melts or in the form of solutions, in particular for the preparation of spinning solutions, paint systems, coating materials, inks or printing inks.

In a particularly preferred embodiment of this invention, the inventive compositions are used for the mass coloration of polyvinyl chloride, polyamides and, especially, polyolefins such as polyethylene and polypropylene as well as for the preparation of paint systems, including powder coatings, inks, printing inks, color filters and coating colors.

Illustrative examples of preferred binders for paint systems are alkyd/melamine resin paints, acryl/melamine resin paints, cellulose acetate/cellulose butyrate paints and two-pack system lacquers based on acrylic resins which are crosslinkable with polyisocyanate.

According to observations made to date, the inventive compositions can be added in any desired amount to the material to be coloured, depending on the end use requirements.

Hence, another embodiment of the present invention relates to a composition comprising
(a) 0.01 to 50, preferably 0.01 to 5, particularly preferred 0.01 to 2% by weight, based on the total weight of the coloured high molecular organic material, of a composition according to the present invention, and
(b) 99.99 to 50, preferably 99.99 to 95, particularly preferred 99.99 to 98% by weight, based on the total weight of the coloured high molecular organic material, of a high molecular organic material, and
(c) optionally, customary additives such as rheology improvers, dispersants, fillers, paint auxiliaries, siccatives, plasticizers, UV-stabilizers, and/or additional pigments or corresponding precursors in effective amounts, such as e.g. from 0 to 50% by weight, based on the total weight of (a) and (b).

To obtain different shades, the inventive fluorescent DPP compounds of formula I may advantageously be used in admixture with fillers, transparent and opaque white, colored and/or black pigments as well as customary luster pigments in the desired amount.

For the preparation of paints systems, coating materials, color filters, inks and printing inks, the corresponding high molecular weight organic materials, such as binders, synthetic resin dispersions etc. and the inventive compositions are usually dispersed or dissolved together, if desired together with customary additives such as dispersants, fillers, paint auxiliaries, siccatives, plasticizers and/or additional pigments or pigment precursors, in a common solvent or mixture of solvents. This can be achieved by dispersing or dissolving the individual components by themselves, or also several components together, and only then bringing all components together, or by adding everything together at once.

Hence, a further embodiment of the present invention relates to a method of using the inventive compositions for the preparation of dispersions and the corresponding dispersions, and paint systems, coating materials, color filters, inks and printing inks comprising the inventive compositions.

A particularly preferred embodiment relates to the use of the inventive compositions for the preparation of fluorescent tracers for e.g. leak detection of fluids such as lubricants, cooling systems etc., as well as to fluorescent tracers or lubricants comprising the inventive compositions.

For the pigmentation of high molecular weight organic material, the inventive compositions, optionally in the form of masterbatches, are mixed with the high molecular weight organic materials using roll mills, mixing apparatus or grinding apparatus. Generally, the pigmented material is subsequently brought into the desired final form by conventional processes, such as calandering, compression molding, extrusion, spreading, casting or injection molding.

For pigmenting lacquers, coating materials and printing inks the high molecular weight organic materials and the inventive compositions, alone or together with additives, such as fillers, other pigments, siccatives or plasticizers, are generally dissolved or dispersed in a common organic solvent or solvent mixture. In this case it is possible to adopt a procedure whereby the individual components are dispersed or dissolved individually or else two or more are dispersed or dissolved together and only then are all of the components combined.

The present invention additionally relates to inks comprising a coloristically effective amount of the pigment dispersion of the inventive compositions.

The weight ratio of the pigment dispersion to the ink in general is chosen in the range of from 0.001 to 75% by weight, preferably from 0.01 to 50% by weight, based on the overall weight of the ink.

The preparation and use of color filters or color-pigmented high molecular weight organic materials are well-known in the art and described e.g. in Displays 14/2, 1151 (1993), EP-A 784085, or GB-A 2,310,072.

The color filters can be coated for example using inks, especially printing inks, which can comprise pigment dispersions comprising the inventive compositions or can be prepared for example by mixing a pigment dispersion comprising an inventive composition with chemically, thermally or photolytically structurable high molecular weight organic material (so-called resist). The subsequent preparation can be carried out, for example, in analogy to EP-A 654 711 by application to a substrate, such as a LCD (liquid crystal display), subsequent photostructuring and development.

Particular preference for the production of color filters is given to pigment dispersions comprising an inventive composition which possess non-aqueous solvents or dispersion media for polymers.

The present invention relates, moreover, to toners comprising a pigment dispersion containing an inventive composition or a high molecular weight organic material pigmented with an inventive composition in a coloristically effective amount.

The present invention additionally relates to colorants, colored plastics, polymeric ink particles, or non-impact-printing material comprising an inventive composition, preferably in the form of a dispersion, or a high molecular weight organic material pigmented with an inventive composition in a coloristically effective amount.

A coloristically effective amount of the pigment dispersion according to this invention comprising an inventive composition denotes in general from 0.0001 to 99.99% by weight, preferably from 0.001 to 50% by weight and, with particular preference, from 0.01 to 50% by weight, based on the overall weight of the material pigmented therewith.

The inventive compositions can be applied to colour polyamides, because they do not decompose during the incorporation into the polyamides. Further, they exhibit an exceptionally good lightfastness, a superior heat stability, especially in plastics.

The organic EL device of the present invention has significant industrial values since it can be adapted for a flat panel display of an on-wall television set, a flat light-emitting device, a light source for a copying machine or a printer, a light source for a liquid crystal display or counter, a display signboard and a signal light. The compositions of the present invention can be used in the fields of an organic EL device, an electrophotographic photoreceptor, a photoelectric converter, a solar cell, an image sensor, and the like.

The following examples illustrate various embodiments of the present invention, but the scope of the invention is not limited thereto. In the examples the “parts” denote “parts by weight” and the “percentages” denote “percentages by weight”, unless otherwise stated.

### EXAMPLES

### Example 1

2.03 g (6.4 mmol) of 1,4-diketo-3,6-bis(4-metrhylphenyl)pyrrolopyrrole are slurred in 1-methyl 2-pyrrolidinone for 2 hours at room temperature. 1.31 g (11.53 mmol) of potassium t-butoxide are added to the slurry under nitrogen. After stirring for 2 hours, 20.5 g (11.1 mmol) of 1-bromoethylbenzene are added to the reaction mixture and agitated additional 2 hours. Then, the mixture is poured into 50 ml of water and the precipitate is collected by filtration and purified by column chromatography (silica gel, dichloromethane as eluent), followed by washing with methanol. After drying 780 mg of a fluorescent orange solid is obtained (mp.=262° C., yield: 24%).

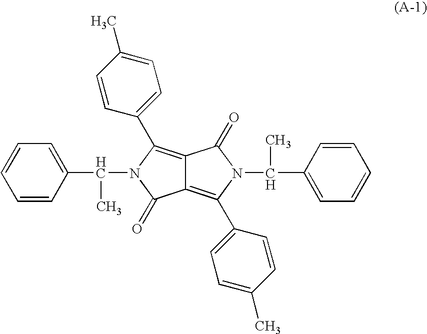

### Example 2

Example 1 was repeated except that 1,4-diketo-3,6-bis(1-naphtyl)-pyrrolo-(3,4-c)-pyrrole was used as starting material. Orange solid (mp.=263° C., yield: 32%).

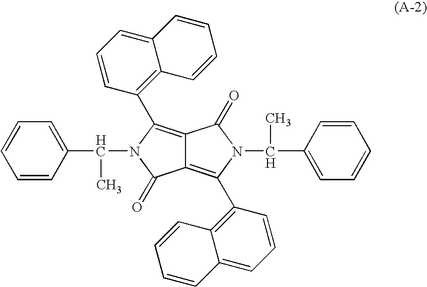

### Example 3

(1.0 mmol) of 2,5-di-benzyl-1,4-diketo-3,6-(4-bromophenyl)pyrrolo(3,4,-c)pyrrole, 2.5 mmol of di-tolylamine, 5 mg of Palladium(II)acetate, 1 mg of tert-butylphosphine and 50 ml of dry xylene were placed in a three necked flask and stirred at 120° C. under nitrogen for 13 hours. After the completion of the reaction xylene was removed under reduced pressure and the residue was purified by column chromatography (silica gel, dichloromethan as eluent). After drying 0.4 g of the desired product was obtained as red solid (mp.=395° C.).

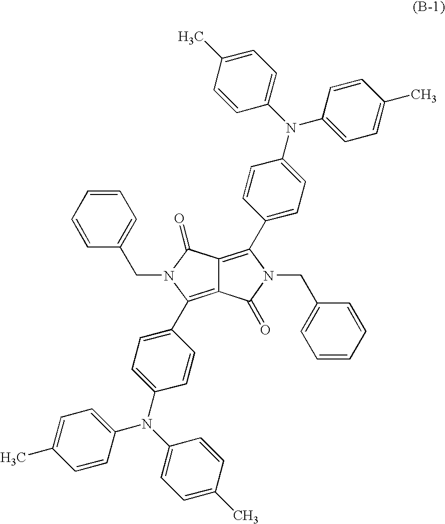

### Example 4

Example 3 is repeated, except that 2,5-di-butyl-1,4-diketo-3,6-(4-bromophenyl)pyrrolo(3,4-c)pyrrole is used as starting material and bis(2-naphtyl)amine is used as reagent, whereby a red solid is obtained (mp.=222-224° C., yield: 46%).

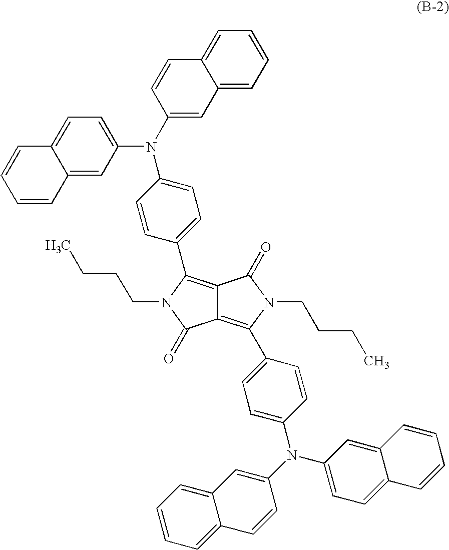

### Example 5

Example 3 is repeated, except that 2-naphtylphenylamine is used instead of di-tolylamine, whereby a red solid is obtained (mp.=361° C., yield: 53%).

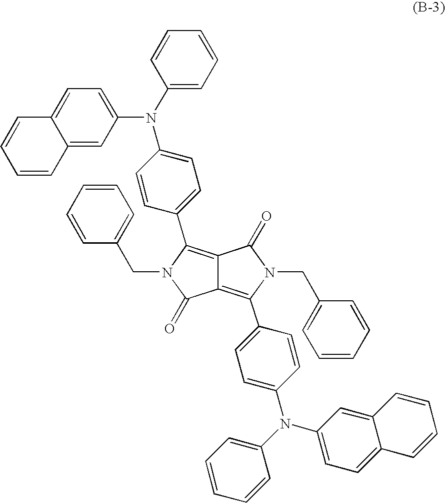

### Example 6

A glass substrate (manufactured by Asahi Glass Co., a product prepared by electron beam vapor deposition method) on which an ITO transparent electroconductive film had been deposited up to a thickness of ca. 210 nm is cut into a size of 30×40 mm, and etched. The substrate thus obtained is subjected to ultrasonic washing with acetone for 15 minutes and then to ultrasonic washing with Semikoklin 56 for 15 minutes, and then washing with ultra-pure water. Subsequently, the substrate is subjected to ultrasonic washing with isopropyl alcohol for 15 minutes, dipped in hot methanol for 15 minutes, and then dried. Just before forming the substrate into an element, the substrate thus obtained is subjected to an UV-ozone treatment for one hour and placed in a vacuum vapour deposition apparatus, and the apparatus is evacuated until the inner pressure reached 1×10⁻⁵ Pa or less. Then, according to the resistance heating method, N,N′-diphenyl-N,N′-(3-methylphenyl)-1,1′-diphenyl-4,4′-diamine (TPD) is vapor-deposited as a positive hole transporting material up to a thickness of 50 nm, to form a positive hole transporting layer. Subsequently, the DPP compounds obtained in example 1 (A-1) and example 6 (B-4) are co-deposited as a light emitting layer up to a thickness of 50 nm by controlling the ratio of deposition rate (A-1: B-4=99: ca. 1) to form an uniform light emitting layer. Subsequently, a Alq₃ layer is vapor-deposited to form an electron transporting layer having a thickness of 50 nm. On top of that, a Mg—Ag alloy (10:1) is vapor-deposited to form a cathode having a thickness of 150 nm, whereby an element having a size of 5×5 mm square is prepared.

The luminescent peak wavelength and emission intensity of the luminescent element thus obtained is summarized in Table 1.

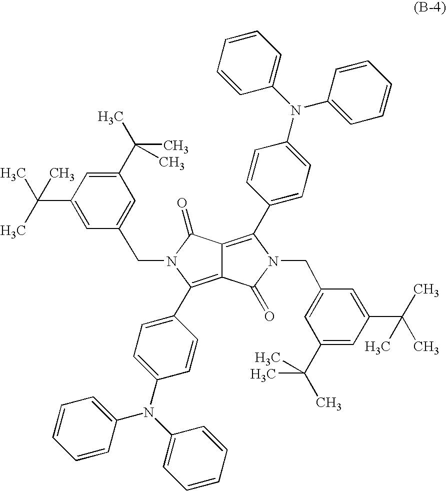

### Example 104 of EP-A-1087006

### Example 7, 8, 9 and 10

Example 6 is repeated, except that the emitting material of example 6 is replaced by the emitting materials as described in table 1.

TABLE 1

| | Emitting Material | | | |
|---|---|---|---|---|
| | Compound of | Compound of | EL properties | |
| Device of | formula I | formula II | | Intensity |
| Example | [99 wt %] | [ca. 1 wt %] | Peak (nm) | (cd/m2) |
| Ex. 6 | A-1 | B-4 | 590 | 10980 |
| Ex. 7 | A-1 | B-1 | 608 | 9026 |
| Ex. 8 | A-2 | B-1 | 610 | 9216 |
| Ex. 9 | A-2 | B-2 | 594 | 6773 |
| Ex. 10 | A-2 | B-3 | 600 | 12260 |
| Reference | A-3 | — | 566 | 5260 |
| Example 1 | (100%) | | | |
| Reference | A-1 | — | 534 | 2600 |
| Example 2 | (100%) | | | |

### Example 11

Example 2 was repeated except that 3-(1-bromoethyl)-1-tert-butylbenzene was used instead of 1-bromoethylbenzene. Orange solid (mp.=307° C., yield: 18%).

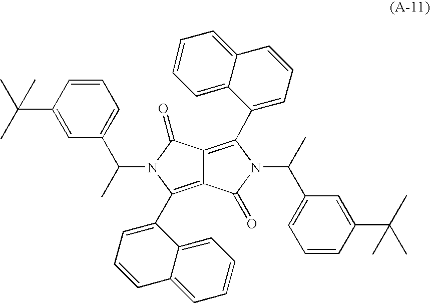

### Example 12

Example 2 was repeated except that 3-(1-bromoethyl)toluene was used instead of 1-bromoethylbenzene. Orange solid (mp.=243° C., yield: 14%).

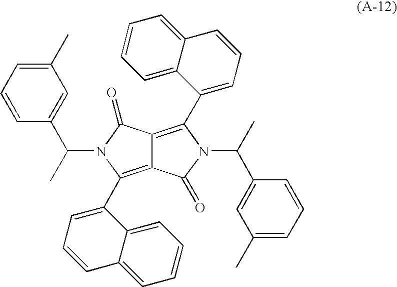

### Example 13

Example 2 was repeated except that 2-(1-bromoethyl)naphthalene was used instead of 1-bromoethylbenzene. Orange solid (mp.=325-329° C., yield: 10%).

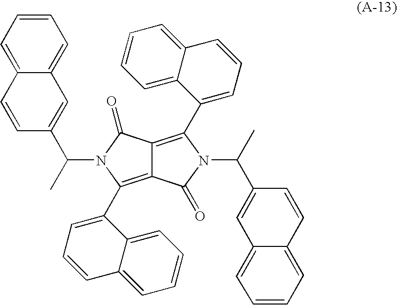

### Example 14

Example 2 was repeated except that 1-(1-bromoethyl)naphthalene was used instead of 1-bromoethylbenzene. Orange solid (mp.=266° C., yield: 17%).

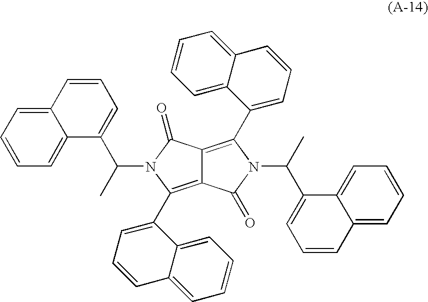

### Example 15

Example 2 was repeated except that 4-bromo-1-(1-bromoethyl)benzene was used instead of 1-bromoethylbenzene. Orange solid (mp.=223-225° C., yield: 32%).

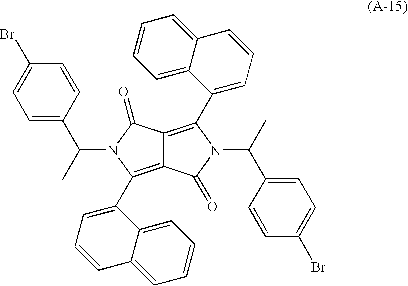

### Example 16

Example 2 was repeated except that 4-phenyl-1-(1-bromoethyl)benzene was used instead of 1-bromoethylbenzene. Orange solid (mp.=293° C., yield: 16%).

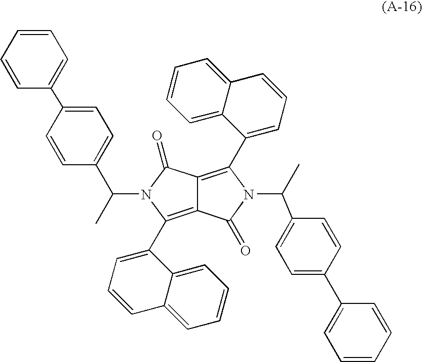

### Example 17

Example 2 was repeated except that isopropyl iodide was used instead of 1-bromoethylbenzene. Orange solid (mp.=294-295° C., yield: 3%).

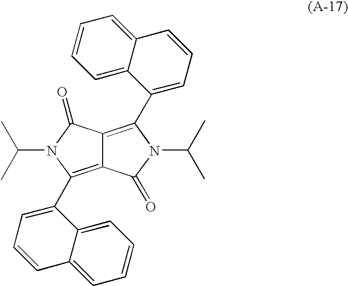

### Example 18

Example 2 was repeated except that 1-bromo-1,2,3,4-tetrahydronaphthalene was used instead of 1-bromoethylbenzene. Orange solid (mp.=360° C., yield: 3%).

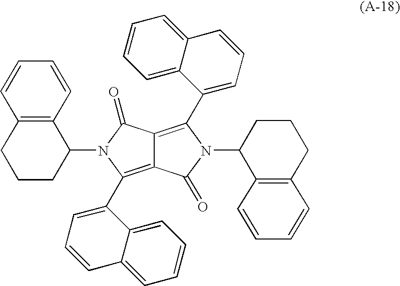

### Example 19

Example 2 was repeated except that bromo d-phenyl methane was used instead of 1-bromoethylbenzene. Orange solid (mp.=258-266° C., yield: 11%).

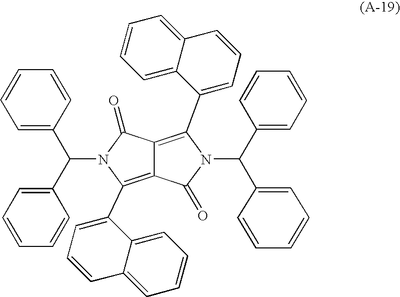

### Example 20

Example 1 was repeated except that 1,4-diketo-3,6-bis(1-phenanthrenyl)-pyrrolo-(3,4-c)-pyrrole was used as starting material. Orange solid (mp.=326° C., yield: 4%).

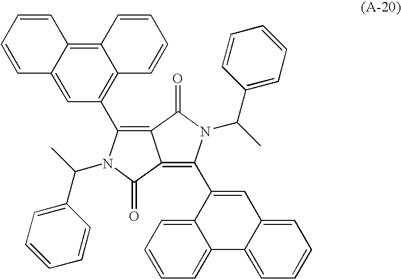

### Example 21

Example 3 was repeated except that 2,5-bis-(4-cyanobenzyl)-1,4-diketo-3,6-(4-bromophenyl)pyrrolo(3,4,-c)pyrrole and 1,1′-bis(diphenylphosphino)ferrocene were used as a starting material and Pd-ligand, respectively. Red violet solid (mp.=376° C., yield: 45%).

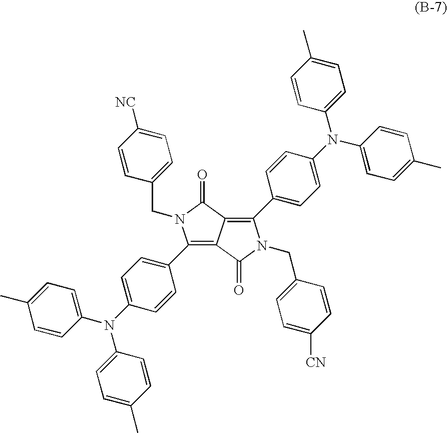

### Example 22

Example 16 was repeated except that 2,5-bis-(4,5-di-cyanobenzyl)-1,4-diketo-3,6-(4-bromophenyl)pyrrolo(3,4,-c)pyrrole was used as a starting material. Red violet solid (mp.=353-356° C., yield: 17%).

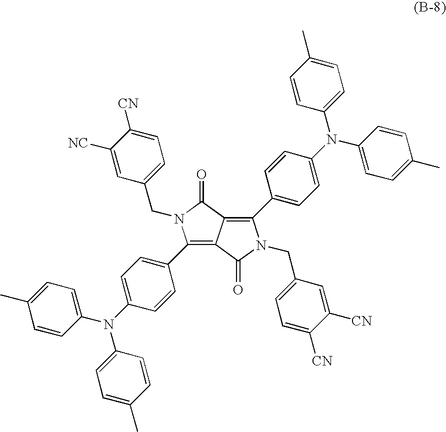

### Example 23

Example 4 was repeated except that benzylbromide was used instead of iodobutane Red violet solid (mp.=359-361° C., yield: 12%).

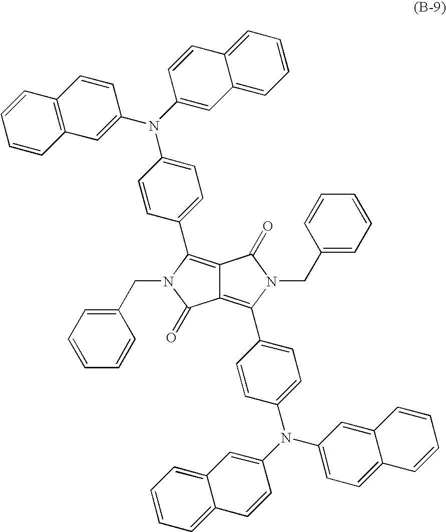

### Example 24-26

Example 6 is repeated, except that the emitting material of example 6 is replaced by the emitting materials as described in table 2.

TABLE 2

| | Emitting Material | | | |
|---|---|---|---|---|
| | Compound of | Compound of | EL properties | |
| Device of | formula I | formula II | | Intensity |
| Example | (wt %) | (wt %) | Peak (nm) | (cd/m²) |
| Ex. 24 | A-2 (97.3) | B-7 (2.7) | 624 | 2717 |
| Ex. 25 | A-11 (97.5) | B-1 (2.5) | 610 | 3996 |
| Ex. 26 | A-11 (97.3) | B-7 (2.7) | 614 | 5074 |

### Example 27

Example 2 was repeated except that 2-(i-bromoethyl)toluene was used instead of 1-bromoethylbenzene. Yellow solid (mp.=276-278° C., yield: 9%).

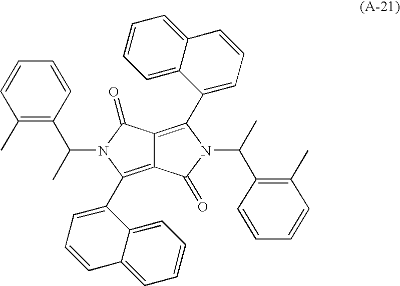

### Reference Example 1

Example 8 is repeated, except that the compound below (A-3; Example 81 of EP-A-1087006) is used as the light emitting material. The maximum luminance is 5260 Cd/m².

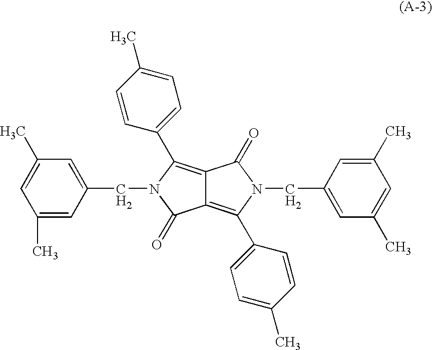

### Reference Example 2

Example 6 is repeated, except that A-1 (Example 93 of EP-A-1087006) is used as the light emitting material. The maximum luminance thereof is 2600 Cd/m².

As evident from the examples the composition of the present invention, comprising a DPP of the formula I and a DPP of the formula II, can provide a luminescent element which is high in the efficiency of electrical energy utilisation and is characterized by a much higher luminance than the individual DPP compounds of formula I and II.

### Example 28

### Film Preparation of Color Changing Media

8 mg of A-2, 2 mg of B-7, 1 g of PMMA (Produced by Wako Pure Chemical Industries, Ltd.) are put in a bottle, and the mixture is dissolved in 5 g of toluene. The solution is dropped on a slid glass substrate, and coated on the glass by use of a spin coater with a rotating rate of 500 rpm for 30 seconds. The obtained film is dried over 80° C. and a CCM film is obtained. The film is evaluated by use of fluorescence spectrophotometer F-4500 (Hitachi, Ltd.). When the film is irradiated by blue light with 470 nm, the film emits red light, the peak of which locates at 597 nm. Thus, the composition comprising the host and the guest is found to be applicable to CCM converting effectively blue light into red light.

1. A composition comprising a guest chromophore and a host chromophore, wherein the absorption spectrum of the guest chromophore overlaps with the fluorescence emission spectrum of the host chromophore, wherein the host chromophore is a diketopyrrolopyrrole having a photoluminescence emission peak at 500 to 720 nm, and wherein the guest chromophore is a diketopyrrolopyrrole having an absorption peak at 500 to 720 nm.

2. A composition according to claim 1, wherein the host chromophore is a diketopyrrolopyrrole (“DPP”) represented by formula I 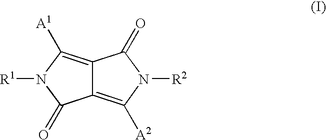
and the guest chromophore is a DPP represented by formula II 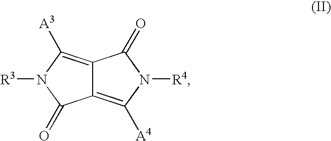
wherein R¹, R², R³ and R⁴ independently from each other stand for C₁-C₂₅-alkyl, which can be substituted by fluorine, chlorine or bromine, C₅-C₁₂-cycloalkyl or C₅-C₁₂-cycloalkyl which can be condensed one or two times by phenyl which can be substituted one to three times with C₁-C₄-alkyl, halogen, nitro or cyano, silyl, A⁵ or CR¹¹R¹² (CH₂)ₘ-A⁵, wherein R¹¹ and R¹² independently from each other stand for hydrogen, fluorine, chlorine, bromine, cyano or C₁-C₄alkyl, which can be substituted by fluorine, chlorine or bromine, or phenyl which can be substituted one to three times with C₁-C₃alkyl, A⁵ stands for phenyl or 1- or 2-naphthyl which can be substituted one to three times with C₁-C₈alkyl, C₁-C₈alkoxy, halogen, nitro, cyano, phenyl, which can be substituted with C₁-C₈alkyl or C₁-C₈alkoxy one to three times, —NR¹³R¹⁴ wherein R¹³ and R¹⁴ represent hydrogen, C₁-C₂₅-alkyl, C₅-C₁₂-cycloalkyl or C₆-C₂₄-aryl, in particular phenyl or 1- or 2-naphthyl which can be substituted one to three times with C₁-C₈alkyl, C₁-C₈alkoxy, halogen or cyano, or phenyl, which can be substituted with C₁-C₈alkyl or C₁-C₈alkoxy one to three times, and m stands for 0, 1, 2, 3 or 4,
A¹ and A² independently from each other stand for 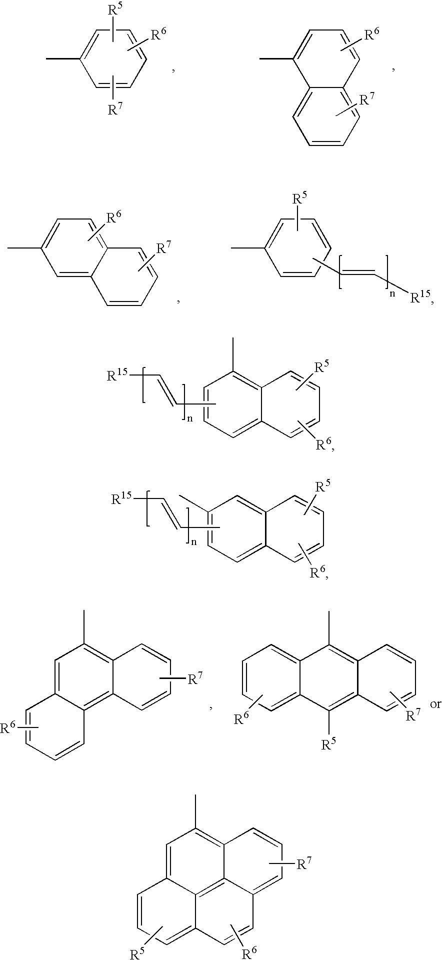
wherein
R⁵, R⁶, R⁷ independently from each other stands for hydrogen, C₁-C₂₅-alkyl, C₁-C₂₅-alkoxy, —CR¹¹R¹²—(CH₂)ₘ-A⁵, cyano, halogen, —OR¹⁰, —S(O)ₚR¹³, or phenyl, which can be substituted one to three times with C₁-C₈alkyl or C₁-C₈alkoxy, wherein R¹⁰ stands for C₆-C₂₄-aryl, or a saturated or unsaturated heterocyclic radical comprising five to seven ring atoms, wherein the ring consists of carbon atoms and one to three hetero atoms selected from the group consisting of nitrogen, oxygen and sulfur, R¹³ stands for C₁-C₂₅-alkyl, C₅-C₁₂-cycloalkyl, —CR¹¹R¹²_(CH₂)ₘ—Ph, R¹⁵ stands for C₆-C₂₄-aryl, p stands for 0, 1, 2 or 3 and n stands for 0, 1, 2, 3 or 4,
A³ and A⁴ independently from each other stand for 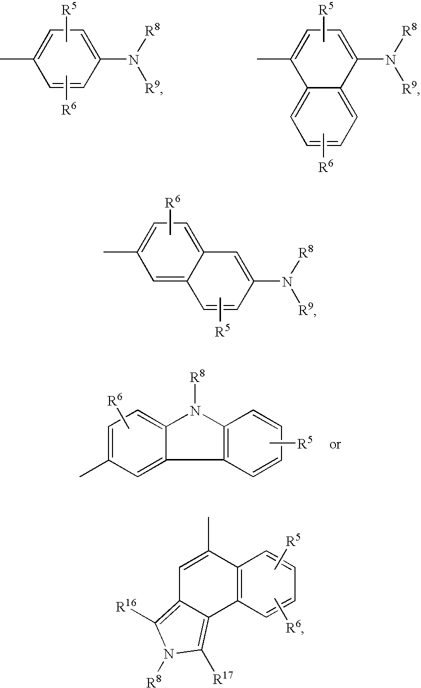
wherein R⁸ and R⁹ independently from each other stand for hydrogen, C₁-C₂₅-alkyl, C₅-C₁₂-cycloalkyl, —CR¹¹R¹²_(CH₂)ₘ-A⁵, C₆-C₂₄-aryl, in particular A¹, or a saturated or unsaturated heterocyclic radical comprising five to seven ring atoms, wherein the ring consists of carbon atoms and one to three hetero atoms selected from the group consisting of nitrogen, oxygen and sulfur, and R¹⁶ and R¹⁷ are independently of each other hydrogen or C₆-C₂₄aryl.

3. Composition according to claim 2, wherein A¹ and A² independently from each other stand for 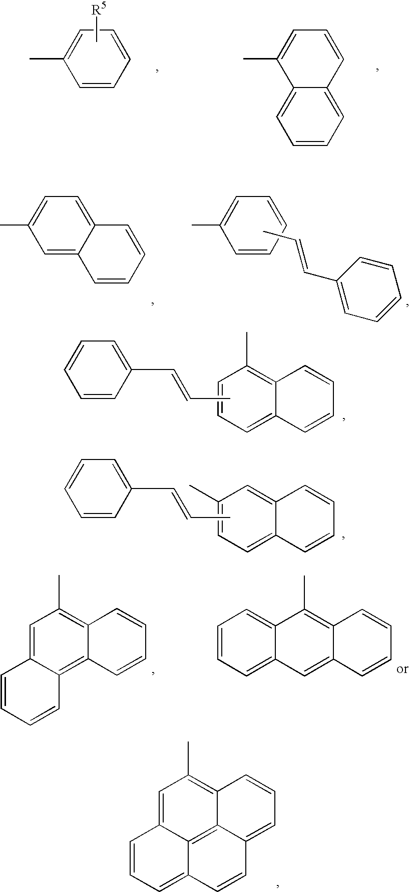
wherein R⁵ is C₁-C₈-alkyl.

4. Composition according to claim 2, wherein A³ and A⁴ independently from each other stand for 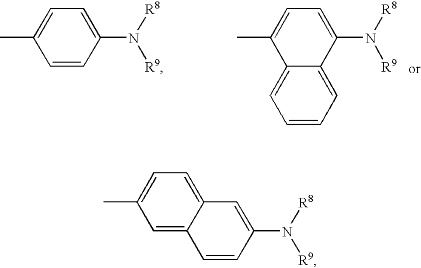
wherein R⁸ and R⁹ independently from each other stand for 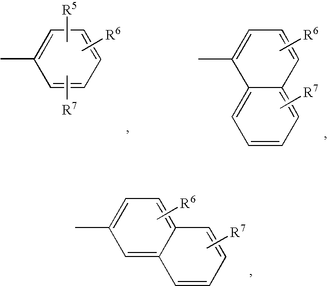
wherein R⁵, R⁶, R⁷ independently from each other for hydrogen, C₁-C₈-alkyl or C₁-C₈-alkoxy.

5. Composition according to claim 2, wherein R¹, R², R³ and R⁴ indendently from each other stand for C₁-C₈alkyl, C₅-C₁₂-cycloalkyl, which can be substituted one to three times with C₁-C₈alkyl and/or C₁-C₈alkoxy, phenyl or 1- or 2-naphthyl which can be substituted one to three times with C₁-C₈alkyl and/or C₁-C₈alkoxy, or —CR¹¹R¹²(CH₂)ₘ-A⁵ wherein R¹¹ and R¹² stand for hydrogen, A⁵ stands for phenyl or 1- or 2-naphthyl, which can be substituted one to three times with C₁-C₈alkyl and/or C₁-C₈alkoxy, and m stands for 0 or 1.

6. Composition according to claim 2, wherein the compound of the formula I is selected from the following compounds A-1 to A-29:

| | |
|---|---|
| | (I) |
| 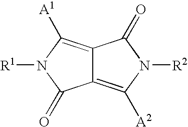 | |
| Compound | A¹ = A² | R¹ = R² |
|---|---|---|
| A-1 | 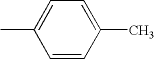 | 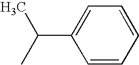 |
| A-2 | 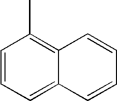 | 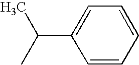 |
| A-3 | 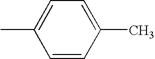 | 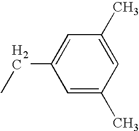 |
| A-4 | 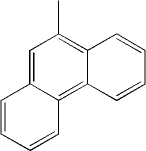 | 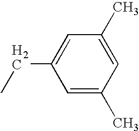 |
| A-5 | ″ | 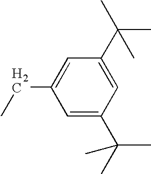 |
| A-6 | ″ | —(CH₂)₃CH₃ |
| A-7 | 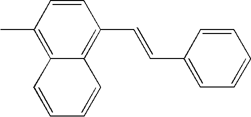 | 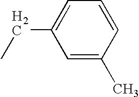 |
| A-8 | 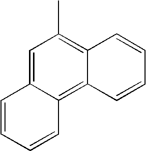 | —Si(CH₃)₃ |
| A-9 | 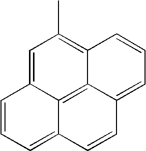 | 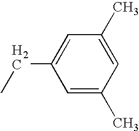 |
| A-10 | 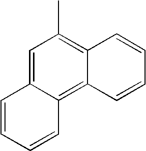 | 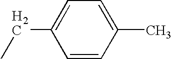 |
| A-11 | 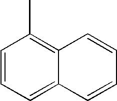 | 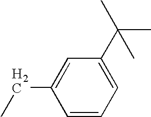 |
| A-12 | 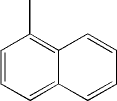 | 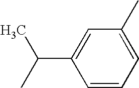 |
| A-13 | 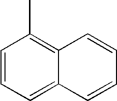 | 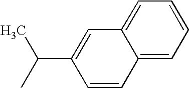 |
| A-14 | 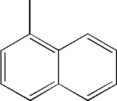 | 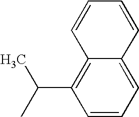 |
| A-15 | 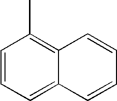 | 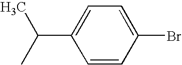 |
| A-16 | 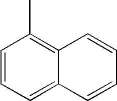 | 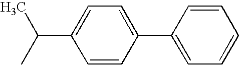 |
| A-17 | 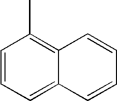 | —CH(CH₃)₂ |
| A-18 | 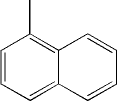 | 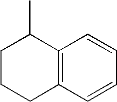 |
| A-19 | 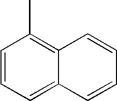 | 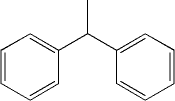 |
| A-20 | 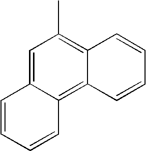 | 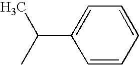 |
| A-21 | 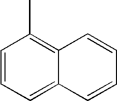 | 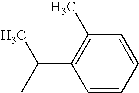 |
| A-22 | 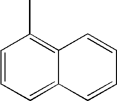 | 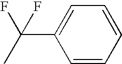 |
| A-23 | ″ | 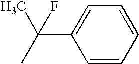 |
| A-24 | ″ | —CF₃ |
| A-25 | ″ | —CHF₂ |
| A-26 | ″ | —CH₂F |
| A-27 | ″ | 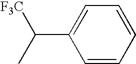 |
| A-28 | ″ | 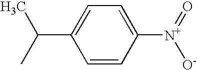 |
| A-29 | ″ | 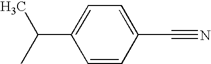 |

7. Composition according to claim 2, wherein the compound of the formula II is selected from the following compounds B-1 to B-9:

| | |
|---|---|
| | (II) |
| 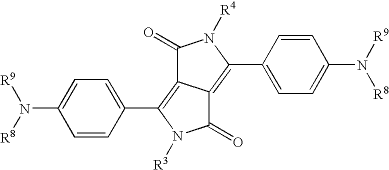 | |
| Compound | R³ = R⁴ | R⁸ | R⁹ |
|---|---|---|---|
| B-1 | 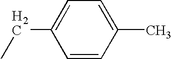 | 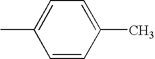 | 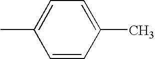 |
| B-2 | 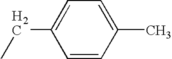 | 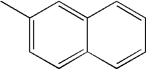 | 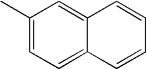 |
| B-3 | 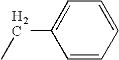 | 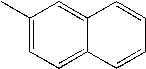 | 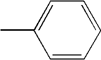 |
| B-4 | 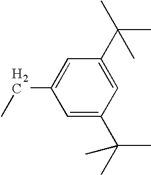 | 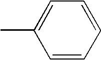 | 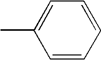 |
| B-5 | 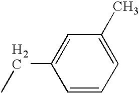 | ″ | ″ |
| B-6 | ″ | 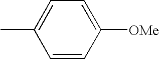 | 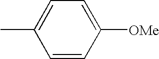 |
| B-7 | 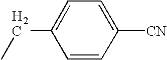 | 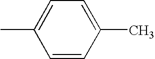 | 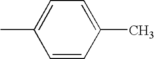 |
| B-8 | 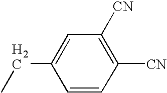 | 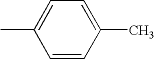 | 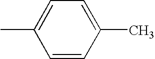 |
| B-9 | 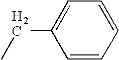 | 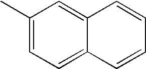 | 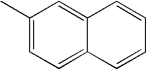 |

8. An electroluminescent device comprising the composition according to claim 1.

9. An electroluminescent device according to claim 8, comprising in this order (a) an anode, (b) a hole transporting layer, (c) a light-emitting layer, (d) optionally an electron transporting layer and (e) a cathode.

10. A composition comprising
(a) 0.01 to 50% by weight, based on the total weight of the colored high molecular weight organic material, of the composition according to claim 1, and
(b) 99.99 to 50% by weight, based on the total weight of the colored high molecular weight organic material, of a high molecular organic material.

11. A method for coloring a high molecular weight organic material or and in color changing media by mixing a compostion according to claim 1 with high molecular weight organic material or media compositions.

12. A diketopyrrolopyrrole (“DPP”) represented by formula I or II 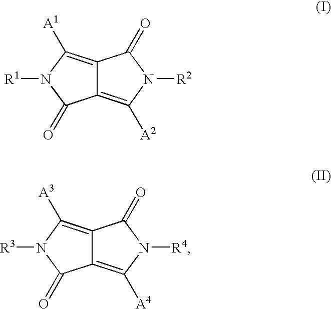
wherein R¹, R², R³ and R⁴ independently from each other stand for C₁-C₂₅-alkyl, which can be substituted by fluorine, chlorine or bromine, C₅-C₁₂-cycloalkyl or C₅-C₁₂-cycloalkyl which can be condensed one or two times by phenyl which can be substituted one to three times with C₁-C₄-alkyl, halogen, nitro or cyano, silyl, A⁵ or —CR¹¹R¹² (CH₂)ₘ-A⁵, wherein R¹¹ and R¹² independently from each other stand for hydrogen, fluorine, chlorine, bromine, cyano or C₁-C₄alkyl, which can be substituted by fluorine, chlorine or bromine, or phenyl which can be substituted one to three times with C₁-C₃alkyl, A⁵ stands for phenyl or 1- or 2-naphthyl which can be substituted one to three times with C₁-C₈alkyl, C₁-C₈alkoxy, halogen, nitro, cyano, phenyl, which can be substituted with C₁-C₈alkyl or C₁-C₈alkoxy one to three times, —NR¹³R¹⁴ wherein R¹³ and R¹⁴ represent hydrogen, C₁-C₂₅-alkyl, C₅-C₁₂-cycloalkyl or C₆-C₂₄-aryl, in particular phenyl or 1- or 2-naphthyl which can be substituted one to three times with C₁-C₈alkyl, C₁-C₈alkoxy, halogen or cyano, or phenyl, which can be substituted with C₁-C₈alkyl or C₁-C₈alkoxy one to three times, and m stands for 0, 1, 2, 3 or 4,
A¹ and A² independently from each other stand for 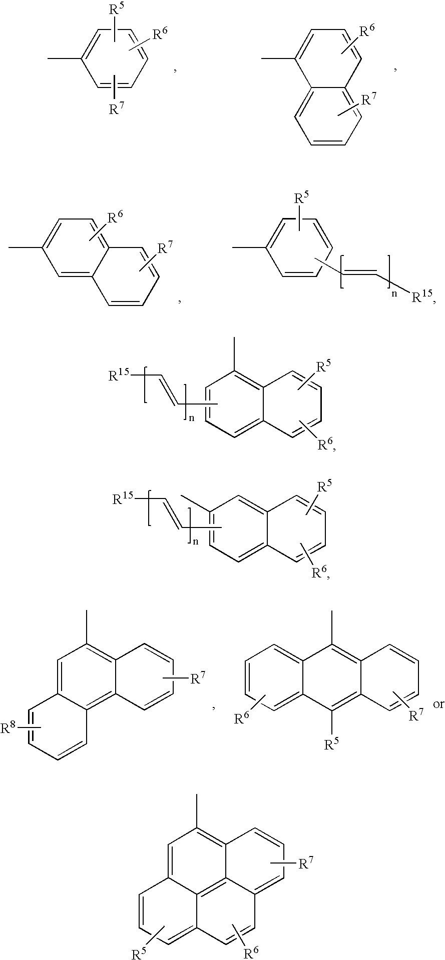
wherein
R⁵, R⁶, R⁷ independently from each other stands for hydrogen, C₁-C₂₅-alkyl, C₁-C₂₅-alkoxy, —CR¹¹R¹²(CH₂)ₘ-A⁵, cyano, halogen, —OR¹⁰, —S(O)ₚR¹³, or phenyl, which can be substituted one to three times with C₁-C₈alkyl or C₁-C₈alkoxy, wherein R¹⁰ stands for C₆-C₂₄-aryl, or a saturated or unsaturated heterocyclic radical comprising five to seven ring atoms, wherein the ring consists of carbon atoms and one to three hetero atoms selected from the group consisting of nitrogen, oxygen and sulfur, R¹³ stands for C₁-C₂₅-alkyl, C₅-C₁₂-cycloalkyl, —CR¹¹R¹²—(CH₂)ₘ—Ph, R¹⁵ stands for C₆-C₂₄-aryl, p stands for 0, 1, 2 or 3 and n stands for 0, 1, 2, 3 or 4,
A³ and A⁴ independently from each other stand for 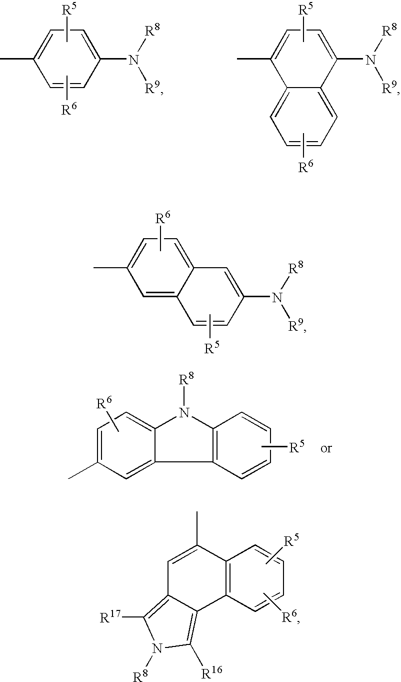
wherein R⁸ and R⁹ independently from each other stand for hydrogen, C₁-C₂₅-alkyl, C₅-C₁₂cycloaolkyl, —CR¹¹R¹²(CH₂)ₘ-A⁵, C₆-C₂₄-aryl, in particular A¹, or a saturated or unsaturated heterocyclic radical comprising five to seven ring atoms, wherein the ring consists of carbon atoms and one to three hetero atoms selected from the group consisting of nitrogen, oxygen and sulfur, and R¹⁶ and R¹⁷ are independently of each other hydrogen or C₆-C₂₄aryl.
